SEQUENCE LISTING (89 sequences)

SEQ ID NO: 1 - protein, 116 aa, Artificial Sequence
  Description of Artificial Sequence Synthetic polypeptide
QVQLVQSGGGVVQPGRSLRLACAASGLTVNNYAMHWVRQAPGKGLEWVAVISYDGNNKYYADSVKGRFTISRDNSKNTLYLQMNSLRAEDTAVYYCAKGGGYFDLWGRGTLVTVSS

SEQ ID NO: 2 - protein, 116 aa, Artificial Sequence
  Description of Artificial Sequence Synthetic polypeptide
QVQLVQSGGGVVQPGRSLRLACAASGFTVNNYAMHWVRQAPGKGLEWVAVISYDGNNKYYADSVKGRFTISRDNSKNTLYLQMNSLRAEDTAVYYCAKGGGYFDLWGRGTLVTVSS

SEQ ID NO: 3 - protein, 116 aa, Artificial Sequence; identical to SEQ ID NO: 2
  Description of Artificial Sequence Synthetic polypeptide

SEQ ID NO: 4 - protein, 116 aa, Artificial Sequence
  Description of Artificial Sequence Synthetic polypeptide
QVQLQQSGGGVVQPGRSLRLSCAASGFTFSSYAMHWVRQAPGKGLEWVAFIRSDGSKKYYADSVKGRFTISRDNSKNTLYLQMNSLRAEDTAVYYCARHGNYFDSWGQGTLVTVSS

SEQ ID NO: 5 - protein, 120 aa, Artificial Sequence
  Description of Artificial Sequence Synthetic polypeptide
QVQLVESGGGVVQPGRSLRLSCAASGFTFSTYGMHWVRQAPGKGLEWLSFISYDGDEKYYADSVKGRFTISRDNSKNTLYLQMNSLRAEDTAVYWCAKASGYGMGILDYWGQGTLVTVSS

SEQ ID NO: 6 - protein, 121 aa, Artificial Sequence
  Description of Artificial Sequence Synthetic polypeptide
EVQLVESGGGLVQPGGSLRLSCAASGFTFSDYYMSWIRQAPGKGLEWVSYISSSGSTIYYADSVKGRFTISRDNSKNTLYLQMNSLRAEDTAVYYCARDYGRIAAAGRRYWGQGTLVTVSS

SEQ ID NO: 7 - protein, 121 aa, Artificial Sequence
  Description of Artificial Sequence Synthetic polypeptide
QVQLQESGGGVVRPGGSLRLSCAASGFTFSDYYMSWIRQAPGKGLEWVSYISSSGSTIYYADSVKGRFTISRDNSKNTLYLQMNSLRAEDTAVYYCARDYGRIAAAGRHYWGQGTLVTVSS

SEQ ID NO: 8 - protein, 121 aa, Artificial Sequence
  Description of Artificial Sequence Synthetic polypeptide
EVQLVESGGGLVQPGGSLRLSCAASGFTFSDYYMSWIRQAPGKGLEWVSYISSSGSTIYYADSVKGRFTISRDNSKNTLYLQMNSLRAEDTAVYYCARDYGRIAAAGRHYWGQGTLVTVSS

SEQ ID NO: 9 - protein, 127 aa, Artificial Sequence
  Description of Artificial Sequence Synthetic polypeptide
QVQLQESGGGLVQPGGSLRLSCAASGFTFSSYWMSWVRQAPGKGLEWVADIKQDGSEKYYVDSVKGRFTISGDNAKNSLYLQMNSLRAEDTAVYYCAKDVGSTAINYVRAYTWFDPWGQGTLVTVSS

SEQ ID NO: 10 - protein, 122 aa, Artificial Sequence
  Description of Artificial Sequence Synthetic polypeptide
QVQLQESGGGLVQPGGSLRLSCAASGFTFSNYAMSWVRQAPGKGLEWVSTISGSGSSTFYVDSVKGRFTISRDNSKNTLYLQMNSLRAEDTAVYYCAQGLYSSGWANWFDPRGQGTLVTVSS

SEQ ID NO: 11 - protein, 123 aa, Artificial Sequence
  Description of Artificial Sequence Synthetic polypeptide
QVQLQESGGGVVQPGRSLRLSCAASGFTFSSYGMHWVRQAPGKGLEWVAVISYDGSNKYYADSVKGRFTISRDNSKNTLYLQMNSLRAEDTAVYYCAKVMGLAAAGLDAFDIWGQGTTVTVSS

SEQ ID NO: 12 - protein, 117 aa, Artificial Sequence
  Description of Artificial Sequence Synthetic polypeptide
QVQLVQSGGGVVQPGRSLRLSCAASGFTFSSYAMHWVRQAPGKGLEWVAVISYDGSNKYYADSVKGRFTISRDTSTNTLYLQMNSLRADDTAVYYCGRESSGSPGVWGQGTTVTVSS

SEQ ID NO: 13 - protein, 122 aa, Artificial Sequence
  Description of Artificial Sequence Synthetic polypeptide
QVQLVESGGGLIQPGGSLRLSCAASGFTVSSNYMSWVRQAPGKGLEWVSVIYTDGSTYYADSVKGRFTISRDNSKNTLYLQMNSLRAEDTAIYYCARDRGTSGYDWAWFDLWGQGTLVTVSS

SEQ ID NO: 14 - protein, 123 aa, Artificial Sequence
  Description of Artificial Sequence Synthetic polypeptide
QVQLQESGGGLVQPGGSLRLSCAASGFTFSSYAMSWVRQAPGKGLEWVSAISGSGGSTYYADSVKGRFTISRDNSKNTLYMQMNSLRAEDTAVYYCAKDRYYYGSGKDAFDIWGRGTMVTVSS

SEQ ID NO: 15 - protein, 118 aa, Artificial Sequence
  Description of Artificial Sequence Synthetic polypeptide
QVQLVESGGGLVQPGGSLGLSCAASGFTFSNYWMSWVRQAPGKGLEWVANVRQDGGQKYYVDSVKGRFTISRDNAKNSLYLQMNSLRTEDTAVYFCVSQRNSGEHDYWGQGTLVTVSS

SEQ ID NO: 16 - protein, 121 aa, Artificial Sequence; identical to SEQ ID NO: 8
  Description of Artificial Sequence Synthetic polypeptide

SEQ ID NO: 17 - protein, 121 aa, Artificial Sequence
  Description of Artificial Sequence Synthetic polypeptide
EVQLVESGGGLVQPGGSLRLSCAASGFTFSDYYMSWIRQAPGKGLEWVSYISSSGSTIYYADSVKGRFTISRDNSKNTLYLQMNSLRAEDTAVYYCARDYGRIAAAGRNYWGQGTLVTVSS

SEQ ID NO: 18 - protein, 121 aa, Artificial Sequence; identical to SEQ ID NO: 7
  Description of Artificial Sequence Synthetic polypeptide

SEQ ID NO: 19 - protein, 121 aa, Artificial Sequence
  Description of Artificial Sequence Synthetic polypeptide
EVQLVESGGGLVKPGGSLRLSCAASGFTFSDYYMSWIRQAPGKGLEWVSYISSSGSSIYYADSVKGRFTISRDNAKNSLYLQMNSLKAEDTAVYYCARDITDVVGVSFDYWGQGTLVTVSS

SEQ ID NO: 20 - protein, 123 aa, Artificial Sequence
  Description of Artificial Sequence Synthetic polypeptide
EVQLVESGGGVVQPGRSLRLSCAASGFTFSSYGMHWVRQAPGKGLEWVAVISYDGSNKYYADSVKGRFTISRDNSKNTLYLQMNSLRAEDTAEYYCAKVMGLAAAGLDAFDIWGQGTLVTVSS

SEQ ID NO: 21 - protein, 123 aa, Artificial Sequence
  Description of Artificial Sequence Synthetic polypeptide
EVQLVESGGGVVQPGRSLRLSCAASGFTFSSFGMHWVRRAPGKGLEWVAVISYDGSNQYYADSVKGRFTISRDNSKNTLYLQMNSLRAEDTAVYYCGSRPGGGYASGSTVAYWGQGTLVTVSS

SEQ ID NO: 22 - protein, 109 aa, Artificial Sequence
  Description of Artificial Sequence Synthetic polypeptide
QSVLTQPPSVSGAPGQRVTISCTGSSSNIGAGYDVHWYQQLPGTAPKLLIYGNNNRPSGVPDRFSGSKSGTSASLAITGLQAEDEADYYCSSYTSGTWLFGGGTKLTVL

SEQ ID NO: 23 - protein, 109 aa, Artificial Sequence; identical to SEQ ID NO: 22
  Description of Artificial Sequence Synthetic polypeptide

SEQ ID NO: 24 - protein, 109 aa, Artificial Sequence
  Description of Artificial Sequence Synthetic polypeptide
QSVLTQPPSVSGAPGQRVTISCTGSSSNIGAGYDVHWYQQLPGTAPKLLIYGDNNRPSGVPDRFSGSKSGTSASLAITGLQAEDEADYYCSSYTSGTWLFGGGTKLTVL

SEQ ID NO: 25 - protein, 107 aa, Artificial Sequence
  Description of Artificial Sequence Synthetic polypeptide
DIQMTQSPSFLSASVGDRVTITCRASQGISSYLAWYQQKPGKAPKLLIYAASTLQSGVPSSFSGSGSGTEFTLTISSLQPEDFATYYCQQLASYPLTFGGGTKVDIK

SEQ ID NO: 26 - protein, 109 aa, Artificial Sequence
  Description of Artificial Sequence Synthetic polypeptide
SSELTQDPAVSVALGQTVRITCQGDSLRSYYVSWFQQKPGQAPVFVMYGQNNRPSGISERFSGSSSGNTASLIITGAQAEDEADYYCHSRDSSGTHLRVFGGGTKLTVL

SEQ ID NO: 27 - protein, 110 aa, Artificial Sequence
  Description of Artificial Sequence Synthetic polypeptide
QSALTQPPSASATPGQRVTISCSGRTSNIGSNHVYWYQQLPGTAPKLLIYRNNQRPSGVPDRFSGSKSGTSASLAISGLRSEDEADYYCATWDDSLSGEVFGGGTKLTVL

SEQ ID NO: 28 - protein, 109 aa, Artificial Sequence
  Description of Artificial Sequence Synthetic polypeptide
SSELTQDPAVSVALGQTVRITCQGDSLRSYYASWYQQKPGQAPVLVIYGKNNRPSGIPDRFSGSSSGNTASLTITGAQAEDEADYYCNSRDSSGTHLEVFGGGTKVTVL

SEQ ID NO: 29 - protein, 107 aa, Artificial Sequence
  Description of Artificial Sequence Synthetic polypeptide
SSELTQDPAVSVALGQTVRITCQGDTLSTYYANWYQQKPGQAPVLVIYGKNNRPSGIPDRFSGSSSGNTASLTITGAQAEDEADYYCHSRDISGNYLFASGTKLTVL

SEQ ID NO: 30 - protein, 110 aa, Artificial Sequence
  Description of Artificial Sequence Synthetic polypeptide
QSVLTQPPSASGTPGQRVTISCSGSSSNIGSNTVNWSRQLPGTAPKLLIYSNNQRPSGVPDRFSGSKSGTSASLAISGLQSEDEADYYCAAWDDSLNVYVFGTGTKVTVL

SEQ ID NO: 31 - protein, 107 aa, Artificial Sequence
  Description of Artificial Sequence Synthetic polypeptide
KIVLTQSPSSLSASVGDTVTIACRASRDIRNDLAWYQQKPGKAPKLLIYGASSLQSGVPSRFSGSGSGTEFILTISSLQPEDFATYYCHRLNSYPLTFGGGTKVDIK

SEQ ID NO: 32 - protein, 111 aa, Artificial Sequence
  Description of Artificial Sequence Synthetic polypeptide
NFMLTQPASLSGSPGQSITISCTGTSSDVGGYNYVSWYQQHPGYAPKLMIYDVSNRPSGVSNRFSGSKSGNTASLTISGLQAEDEADYYCSSYTSSSTPWVFGGGTKLTVL

SEQ ID NO: 33 - protein, 106 aa, Artificial Sequence
  Description of Artificial Sequence Synthetic polypeptide
SYVLTQDPAVSVALGQTVRITCQGDSLRSYYASWYQQKPGQAPVLVIYGKNNRPSGIPDRFSGSSSGNTASLTITGAQAEDEADYYCNSRDSSGNQFGGGTKLTVL

SEQ ID NO: 34 - protein, 108 aa, Artificial Sequence
  Description of Artificial Sequence Synthetic polypeptide
SSELTQDPAVSVALGQTVRITCQGDSLRTYYASWYQQRPGQAPILVLYGKNNRPSGIPDRFSGSSSGNTASLTITGAQAEDEADYYCNSRDSSGNHVVFGGGTKLTVL

SEQ ID NO: 35 - protein, 110 aa, Artificial Sequence
  Description of Artificial Sequence Synthetic polypeptide
QSVLTQPASVSGSPGQSITISCTGTGSDVGSYNYVSWYQQNPGKAPKLMIYEVSNRPSGVSNRFSGSKSGNTASLTISGLQAEDEADYYCSSYTTSSTLVFGGGTKVTVL

SEQ ID NO: 36 - protein, 107 aa, Artificial Sequence
  Description of Artificial Sequence Synthetic polypeptide
SELTQDPAVSVALGQTVRITCQGDSLRSYYASWYQQKPGQAPVLVIYGENSRPSGIPDRFSGSSSGNTASLTITGAQAEDEADYYCNSWDSSGNHVVFGGGTKLTVL

SEQ ID NO: 37 - protein, 107 aa, Artificial Sequence
  Description of Artificial Sequence Synthetic polypeptide
AIRMTQSPSSLSASVGDRVTITCRASQSISSYLNWYQQKPGKAPKLLIYAASSLQSGVPSRFSGSGSGTDFTLTISSLQPEDFATYYCQQSYSTPRTFGQGTKLEIK

SEQ ID NO: 38 - protein, 111 aa, Artificial Sequence
  Description of Artificial Sequence Synthetic polypeptide
DIVMTQSPLSLPVTPGEPASISCRSSQSLLHSNGYDYLDWYLQKPGQSPQLLIYLGSNRASGVPDRFSGSGSGTDFTLKISRVETEDVGIYYCMQGLQTPSFGQGTKLEIK

SEQ ID NO: 39 - protein, 109 aa, Artificial Sequence
  Description of Artificial Sequence Synthetic polypeptide
SSELTQDPAVSVALGQTVRITCQGDSLRSYYASWYQQKPGQAPVPVIYGKNNRPSGIPDRFSGSSSGNTASLTITGAQAEDEADYYCNSRDSSSTHRGVFGGGTKLTVL

SEQ ID NO: 40 - protein, 107 aa, Artificial Sequence
  Description of Artificial Sequence Synthetic polypeptide
DIQLTQSPSSLSASVGDRVTITCRASRSISTYLSWYQQKPGKAPKLLIYDASRLQNGVPSRFSGSGSDTDFTLTISSLQPEDFATYFCQQSYNPPWTFGQGTKLEIK

SEQ ID NO: 41 - protein, 111 aa, Artificial Sequence
  Description of Artificial Sequence Synthetic polypeptide
QSALTQPASVSGSPGQSITISCTGTSSDVGGYNYVSWYQQHPGKAPKLMIYDVSNRPSGVSNRFSGSKSGNTASLTISGLQAEDEAYYYCSSYTSSSDPWVFGGGTQLTVL

SEQ ID NO: 42 - protein, 107 aa, Artificial Sequence
  Description of Artificial Sequence Synthetic polypeptide
NIQMTQSPSSLSASVGDRVTITCRAGQPISTYVNWYQHKPGKAPKLLIYGASNLQSGVPSRFSGGGSATDFTLTISSLQPEDFATYYCQQSYSSLLTFGDGTKVEIK

SEQ ID NO: 43 - protein, 15 aa, Artificial Sequence
  Description of Artificial Sequence Synthetic peptide
GGGGSGGGGSGGGGS

SEQ ID NO: 44 - protein, 242 aa, Artificial Sequence
  Description of Artificial Sequence Synthetic polypeptide
QVQLQESGGGLVKPGGPLRLSCAASGFTFSSYGMYWVRQAPGKGLEWVSTLSRSGSGTYYADSVKGRFTISRDNSKNTLYLQMNSLRAEDTAVYYCASIAVAGNYFDYWGQGTLVTVSSGGGGSGGGGSGGGGSSYVLTQDPAVSVALGQTVRITCQGDSLRSYYASWYQERPGQAPLLVIYGKNNRPSGIPDRFSGSNSGSTATLTISRVEAGDEGDYYCQVWDSINEQVVFGGGTKVTVL

SEQ ID NO: 45 - protein, 241 aa, Artificial Sequence
  Description of Artificial Sequence Synthetic polypeptide
QVQLVQSGGGVVQPGRSLRLSCAATGIPFSGSGMHWVRQAPGKGLEWVTMIWYDGSNKFYADSVKGRFTISRDNSKNTLYLQMDSLRAEDTAVYFCARDKGVRSMDVWGLGTTVTVSSGGGGSGGGGSGGGGSNFMLTQPPSVSVAPGQTAKITCDGYSIRTKSVHWYQQKPGQAPVVVVHDDSDRPSGIPERFSGSNSGTTATLTISRVEAGDEADYYCQAWDSISEEVVFGGGTKLTVL

SEQ ID NO: 46 - protein, 240 aa, Artificial Sequence
  Description of Artificial Sequence Synthetic polypeptide
QVQLQESGGGLVQPGGSLRLSCSASGFTFGTYAMRWVRQTSGKGLEWVSGIGVSGDAYYTDSVRGRFTISRDNSKNTLYLQMNTLRAEDTATYYCTRKSSTTSNDYWGRGTLVTVSSGGGGSGGGGSGGGGSSYVLTQDPAVSVALGQTVRITCQGDNIGSKSVHWYQQKPGQAPVLVVYDDSDRPSGIPERFSGSNSGTTATLTISSVEAGDEADYYCQAWDSISEHVIFGGGTKVTVL

SEQ ID NO: 47 - protein, 241 aa, Artificial Sequence
  Description of Artificial Sequence Synthetic polypeptide
QVQLQESGGGLVQPGGSLRLSCAASGFTFSSYAMHWVRQAPGKGLEWVAVISYDGSNKYYADSVKGRFTISRDNSKNTLYLQMNSLRAEDTAVYYCARFSSGWYYFDYWGQGTLVTVSSGGGGSGGGGSGGGGSDIQMTQSPSFLSASVGDRITITCRASHDISSYFAWYQQKPGKAPKPLIYAASTLQSGVPSRFSGSGSGTEFTLTISSLQPEDFATYYCQQLGSYPLTFGGGTKLEIK

SEQ ID NO: 48 - protein, 245 aa, Artificial Sequence
  Description of Artificial Sequence Synthetic polypeptide
QVQLLQSGGGLVQPGGSLRLSCAASGFTFSSYAMSWVRQAPGKGLEWVSAISGSGGSTYYTDSVKGRFTISRDNSKNTLYLQMNSLRAEDTAVYYCAKSHDYGDYAGFDYWGQGTLVTVSSGGGGSGGGGSGGGGSHVILTQDPAVSVALGQTVRITCQGDSLKSYYASWYQQKPGQAPVLVIYGKNNRPSGIPDRFSGSSSGTTASLTITGAQAEDEADYYCHSRDSSGTHLRVFGGGTKLTVL

SEQ ID NO: 49 - protein, 244 aa, Artificial Sequence
  Description of Artificial Sequence Synthetic polypeptide
QVQLQESGGGLVKPGGSLRLSCAASGFTFSNAWMNWVRQAPGKGLEWVGRIKSKTDEGTTDYAAPVKGRFSISRDDSKNTLYLQMNSLKTEDTGVYYCTATKGLGGSKLGQGTLVTVSSGGGGSGGGGSGGGGSQSVLTQPPSASGTPGQRVTISCSGSSSNIGNNTVNWSRQLPGTAPKLLIYSNDQRPSGVPDRFSGSKSGTSASLAITGLQPEDEADYYCGTWDSSLSAYVFGTGTKLTVL

SEQ ID NO: 50 - protein, 245 aa, Artificial Sequence
  Description of Artificial Sequence Synthetic polypeptide
QVQLVESGGGVVQPGRSLRLSCAASGFTFSSFGMHWVRRAPGKGLEWVAVISYDGSNQYYADSVKGRFTISRDNSKNTLYLQMNSLRAEDTAVYYCGSRPGGGYASGSTVAYWGQGTPVTVSSGGGGSGGGGSGGGGSSSELTQDPAVSVALGQTVRITCQGDSLRSYYASWYQQKPGQAPLLVIYGQNIRPSGIPDRFSGSSSGNSASLTITGAQAEDEADYYCHSRDSSGKYVFGVGTKVTVL

SEQ ID NO: 51 - protein, 237 aa, Artificial Sequence
  Description of Artificial Sequence Synthetic polypeptide
QVQLVESGGGLVQPGGSLRLSCAASGFTFSSYAMGWVRQAPGKGLEWVSAISGSGGSTYYADSVKGRFTISRDNSKDTLYLQMNSLRAEDTAVYYCASRSLLDYWGQGTLVTVSSGGGGSGGGGSGGGGSNFMLTQDPAVSVALGQTVRITCQGDSLRSYYASWYQQKPGQAPLLVIYGKNNRPSGIPDRFSGSSSGNTASLTITGAQAEDEADYYCNSRDSSGNPVFGGGTKVTVL

SEQ ID NO: 52 - protein, 237 aa, Artificial Sequence
  Description of Artificial Sequence Synthetic polypeptide
QVQLVESGGGLVQPGGSLRLSCAASGFTFSSYAMSWVRQAPGKGLEWVSAISGSGGSTYYADSVKGRFTISRDNSKDTLYLQMNSLRAEDTAVYYCASRSLLDYWGQGTLVTVSSGGGGSGGGGSGGGGSNFMLTQDPAVSVALGQTVRITCQGDSLRSYYASWYQQKPGQAPLLVIYGKNNRPSGIPDRFSGSSSGNTASLTITGAQAEDEADYYCNSRDSSGNPVFGGGTKVTVL

SEQ ID NO: 53 - protein, 243 aa, Artificial Sequence
  Description of Artificial Sequence Synthetic polypeptide
QVQLQESGGGLVQLGGSLRLSCAASGFTFSSYAMSWVRQAPGKGLEWVSAISGSGGSTYYADSVKGRFTISRDNSKNTLYLQMSSLRAEDTAFYYCANSAYTGGWYDYWGHGTLVTVSSGGGGSGGGGSGGGGSSSELTQDPAVSVALGQTVKITCQGDSLRTYYASWYQQRPGQAPVLVIYGENSRPSGIPDRFSGSSSGNTASLTITGAQAEDEADYYCNSRDSSGNHLRVFGGGTKLTVL

SEQ ID NO: 54 - protein, 238 aa, Artificial Sequence
  Description of Artificial Sequence Synthetic polypeptide
QVNLRESGGGLVQPGGFLRLSCAAFGFTFSGYWMSWVHPAPGKGLEWVANIKQDGSEKFYVDSVKGRFTISRDNAKNSLFLQMNSLRAEDTAVYFCARGLLSDYWGQGTLVPVSSGGGGSGGGGSGGGGSNFMLTQPPSVSVAPGKTASLTCGGYNIGTKSVHWYQQKPGQAPVVVVHDDSDRPSGIPERFSGSNSGTTATLTIIRVEAGDEADYYCQAWDSISEEVVFGGGTKLTVL

SEQ ID NO: 55 - protein, 242 aa, Artificial Sequence
  Description of Artificial Sequence Synthetic polypeptide
QVQLQESGGGLVKPGGPLRLSCAASGFTFSSYGMYWVRQAPGKGLEWVSTLSRSGSGTYYAESVKGRFTISRDNSKNTLYFQMNSLRAEDTAVYYCASIAVAGNYFEYWGQGTLVTVSSGGGGSGGGGSGGGGSSYVLTQDPAVSVALGQTVRITCQGDSLRSYYASWYQERPGQAPLLVIYGKNNRPSGIPDRFSGSNSGSTATLTISRVEAGDEGDYYCQVWDSINEQVVFGGGTKVTVL

SEQ ID NO: 56 - protein, 243 aa, Artificial Sequence
  Description of Artificial Sequence Synthetic polypeptide
QVQLQESGGGLVKPGESLRLSCAASGFTFSDHYMDWVRQAPGKGLEWVAYIRYDGSTKYYADSVKGRFTISRDNSKNTLYLQMNSLRPEDTAFYYCARLIAEAEGWFDPWGQGTLVTVSSGGGGSGGGGSGGGGSNFMLTQPPSVSVAPGKTARITCGGNNIGSKSVYWYQQKPGQAPVLVVYDDSDRPSGIPERFSGSNSGNTATLTISRVEAGDEADYYCQVWDSSSDHVVFGGGTKVTVL

SEQ ID NO: 57 - protein, 241 aa, Artificial Sequence
  Description of Artificial Sequence Synthetic polypeptide
QVQLLQSAGGLVQPGGSLRLSCAASGFTFSTYAMNWVRQAPGKGLEWVSGISGSGGSTNYADSVKGRFTISRDSSKNTLFLQMNSLRAEDTAVYYCAKDYGSGWYDYWGQGTLVTVSSGGGGSGGGGSGGGGSSSELTQDPAVSVALGQTVRITCQGDSLRSYYASWYQERPGQAPLLVIYGRNERPSGIPDRFSASSSGNTASLTITGAQAEDEADYYCQVWDSFNEQVVFGGGTKLTVL

SEQ ID NO: 58 - protein, 245 aa, Artificial Sequence
  Description of Artificial Sequence Synthetic polypeptide
QVQLVESGGGLVQPGGSLRLSCAASGFTFSSYAMSWVHQAPGKGLEWVSAISGSGGSTYYADSVKGRFTISRDNSKNTLYLQMNSLRAEDTAVYYCAKTYYGFWSGYYDYLGQGTLVTVSSGGGGSGGGGSGGGGSSSELTQDPAVSVGLGQTVTITCQGDSLRSYYANWYQQKPGQAPILVIYGENNRPSGIPDRFSGSSSGNTASLTITGAQAEDEADYYCHSRDSSGTHLRVFGGGTKLTVL

SEQ ID NO: 59 - protein, 244 aa, Artificial Sequence
  Description of Artificial Sequence Synthetic polypeptide
QVQLLESGGGVVQPGTSLRLSCAASGFTFSNYAINWVRQAAGKGLEWVSGISGSGVSTSYADSVKGRFTVSRDNSKNTLYLQMNSLRVEDTALYYCAKNGGGPEYLQHWGQGTLVTVSSGGGGSGGGGSGGGGSQSVLTQPPSASGTPGQRVTISCSGSSSNIGNNTVNWSRQLPGTAPKLLIYSNDQRPSGVPDRFSGSKSGTSASLAITGLQPEDEADYYCGTWDSSLSAYVFGTGTKLTVL

SEQ ID NO: 60 - protein, 238 aa, Artificial Sequence
  Description of Artificial Sequence Synthetic polypeptide
QVQLQESGGTLVQPGGSLRLSCAASGFTFSSYAMSWVRQAPGRGLEWVSTISGSGGSTYYADSVKGRFTISRDNSKNTLYLQMNSLRAEDTAVYYCAKGAYSGSYWGQGTLVTVSSGGGGSGGGGSGGGGSSSELTQDPAVSVALGQTVRITCQGDSLRSYYASWYQQKPGQAPSLVIYGENSRPSGIPDRFSGSSSGNTASLTITGAQAENEADYYCQAWDSSTAVVFGGGTKLTVL

SEQ ID NO: 61 - protein, 238 aa, Artificial Sequence
  Description of Artificial Sequence Synthetic polypeptide
QVQLQESGGTLVQPGGSLRLSCAASGFTFSSYAMSWVRQAPGRGLEWVSTISGSGGSTYYADSVKGRFTISRDNSKNTLYLQMNSLRAEDTAVYYCAKGAYSGSHWGQGTLVTVSSGGGGSGGGGSGGGGSSSELTQDPAVSVALGQTVRITCQGDSLRSYYASWYQQKPGQAPSLVIYGENSRPSGIPDRFSGSSSGNTASLTITGAQAENEADYYCQAWDSSTAVVFGGGTKLTVL

SEQ ID NO: 62 - protein, 244 aa, Artificial Sequence
  Description of Artificial Sequence Synthetic polypeptide
QVQLVESGAEVKKPGASVKVSCKASGYTFTSYGISWVRQAPGQGLEWMGWISAYNGNTNYAQKLQGRVTMTTDTSTSTAYMELRSLRSDDTAVYYCARPIYDSSGYDAFDIWGQGTMVTVSSGGGGSGGGGSGGGGSDIVMTQSPSTLSASIGDRVTITCRASEGIYHWLAWYQQKPGKAPKLLIYKASSLASGAPSRFSGSGSGTDFTLTISSLQPDDFATYYCQQYHTISRTFGPGTKVDIK

SEQ ID NO: 63 - protein, 244 aa, Artificial Sequence
  Description of Artificial Sequence Synthetic polypeptide
QVQLVESGGGLVKPGGSLRLSCAASGFTFSSYAMHWVRQAPGKGLEWVAVISYDGSNKYYADSVKGRFTISRDNSKNTLYLQMNSLRAEDTAVYFCVRPSDSGWSFEHWGQGTLVPVSSGGGGSGGGGSGGGGSQSVLTQPPSASGTPGQRVTISCSGSSSNIGNNTVNWSRQLPGTAPKLLIYSNDQRPSGVPDRFSGSKSGTSASLAITGLQPEDEADYYCGTWDSSLSAYVFGTGTKLTVL

SEQ ID NO: 64 - protein, 245 aa, Artificial Sequence
  Description of Artificial Sequence Synthetic polypeptide
QVQLQESGGGLVQPGGSLRLSCAASGFTFSSYAMSWVRQAPGKGLEWVAVISYDGSNKYYADSVKGRFTISRDNSKNTLYLQMNSLRAEDTAVYYCVRGDRSYGAEYFQHWGQGTLVTVSSGGGGSGGGGSGGGGSSSELTQDPAVSVASGQTVRITCQGDSLRSYYASWYQQKPGQAPLLVIYGKNIRPSGIPDRFSGSTSGNSASLTITGAQAEDEADYYCNSRDSSGNRNWVFGGGTKLTVL

SEQ ID NO: 65 - protein, 246 aa, Artificial Sequence
  Description of Artificial Sequence Synthetic polypeptide
QVQLQESGGGLVKPGGSLRLSCAASGFTSSSYAMHWVRQAPGKGLEYVSAIGGNGGTYYADSVKGRFTISRDNSKNTLYLQMNSLRAEDTAVYYCAKEGEQWLEYRYYYGMDVWGQGTTVTVSSGGGGSGGGGSGGGGSSSELTQDPAVSVALGQTVRITCQGDSLRSYYASWYQQKPGQAPSLVIYGENSRPSGIPDRFSGSSSGNTASLTITGAQAENEADYYCQAWDSSTAVVFGGGTKLTVL

SEQ ID NO: 66 - protein, 250 aa, Artificial Sequence
  Description of Artificial Sequence Synthetic polypeptide
QVQLVESGGGVVQPGRSLRLSCTASGFTFSSYGMHWVRQAPGKGLEWVAVISYDGSNKYYADSVKGRFTISRDNSKNTLYLQMNSLRAEDTAVYYCARGGRYSSNWFSYYYYGMDVWGQGTTVTVSSGGGGSGGGGSGGGGSNFMLTQPPSVSVAPGKTARITCGGNNIGSKSVYWYQQKPGQAPVLVVYDDSDRPSGIPERFSGSNSGNTATLTISRVEAGDEADYYCQVWDSSSDHVVFGGGTKVTVL

SEQ ID NO: 67 - protein, 15 aa, Artificial Sequence
  Description of Artificial Sequence Synthetic peptide
GGGGSGGGGSGGGGS

SEQ ID NO: 68 - protein, 10 aa, Artificial Sequence
  Description of Artificial Sequence Synthetic peptide
GGGGSGGGGS

SEQ ID NO: 69 - protein, 5 aa, Artificial Sequence
  Description of Artificial Sequence Synthetic peptide
GGGGS

SEQ ID NO: 70 - protein, 20 aa, Artificial Sequence
  Description of Artificial Sequence Synthetic peptide
GSGGGGSGGGGSGGSGGGGS

SEQ ID NO: 71 - protein, 6 aa, Artificial Sequence
  Description of Artificial Sequence Synthetic peptide
SGGGGS

SEQ ID NO: 72 - protein, 4 aa, Artificial Sequence
  Description of Artificial Sequence Synthetic peptide
GGGS

SEQ ID NO: 73 - protein, 4 aa, Artificial Sequence
  Description of Artificial Sequence Synthetic peptide
VPGV

SEQ ID NO: 74 - protein, 5 aa, Artificial Sequence
  Description of Artificial Sequence Synthetic peptide
VPGVG

SEQ ID NO: 75 - protein, 6 aa, Artificial Sequence
  Description of Artificial Sequence Synthetic peptide
GVPGVG

SEQ ID NO: 76 - protein, 8 aa, Artificial Sequence
  Description of Artificial Sequence Synthetic peptide
GVGVPGVG

SEQ ID NO: 77 - protein, 10 aa, Artificial Sequence
  Description of Artificial Sequence Synthetic peptide
VPGVGVPGVG

SEQ ID NO: 78 - protein, 7 aa, Artificial Sequence
  Description of Artificial Sequence Synthetic peptide
GGSSRSS

SEQ ID NO: 79 - protein, 18 aa, Artificial Sequence
  Description of Artificial Sequence Synthetic peptide
GGSSRSSSSGGGGSGGGG

SEQ ID NO: 80 - protein, 8 aa, Artificial Sequence
  Description of Artificial Sequence Synthetic peptide
GLTVNNYA

SEQ ID NO: 81 - protein, 8 aa, Artificial Sequence
  Description of Artificial Sequence Synthetic peptide
ISYDGNNK

SEQ ID NO: 82 - protein, 9 aa, Artificial Sequence
  Description of Artificial Sequence Synthetic peptide
AKGGGYFDL

SEQ ID NO: 83 - protein, 9 aa, Artificial Sequence
  Description of Artificial Sequence Synthetic peptide
SSNIGAGYD

SEQ ID NO: 84 - protein, 3 aa, Artificial Sequence
  Description of Artificial Sequence Synthetic peptide
GNN

SEQ ID NO: 85 - protein, 9 aa, Artificial Sequence
  Description of Artificial Sequence Synthetic peptide
SSYTSGTWL

SEQ ID NO: 86 - protein, 116 aa, Artificial Sequence; identical to SEQ ID NO: 1
  Description of Artificial Sequence Synthetic polypeptide

SEQ ID NO: 87 - protein, 109 aa, Artificial Sequence; identical to SEQ ID NO: 22
  Description of Artificial Sequence Synthetic polypeptide

SEQ ID NO: 88 - protein, 446 aa, Artificial Sequence
  Description of Artificial Sequence Synthetic polypeptide
QVQLVQSGGGVVQPGRSLRLACAASGLTVNNYAMHWVRQAPGKGLEWVAVISYDGNNKYYADSVKGRFTISRDNSKNTLYLQMNSLRAEDTAVYYCAKGGGYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 89 - protein, 215 aa, Artificial Sequence
  Description of Artificial Sequence Synthetic polypeptide
QSVLTQPPSVSGAPGQRVTISCTGSSSNIGAGYDVHWYQQLPGTAPKLLIYGNNNRPSGVPDRFSGSKSGTSASLAITGLQAEDEADYYCSSYTSGTWLFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

(19) United States
(12) Patent Application Publication
(10) Pub. No.: US 2024/0101699 A1
(43) Pub. Date: Mar. 28, 2024

(54) MODULATION OF CD46 CELL SURFACE EXPRESSION AND THERAPEUTIC USE THEREOF

(71) Applicant: The Regents of the University of California, Oakland, CA (US)

(72) Inventors: Bin Liu, San Francisco, CA (US); Yang Su, San Francisco, CA (US); Scott Bidlingmaier, San Francisco, CA (US)

(73) Assignee: The Regents of the University of California, Oakland, CA (US)

(21) Appl. No.: 18/271,205

(22) PCT Filed: Jan. 6, 2022
(86) PCT No.: PCT/US2022/011500

§ 371 Date: Jul. 6, 2023

Related U.S. Application Data
(60) Provisional application No. 63/134,720, filed on Jan. 7, 2021.

(51) Int. Cl. C07K 16/28; A61K 31/454; A61K 31/573; A61K 47/68; A61P 35/00

(52) U.S. Cl. CPC C07K 16/2896; A61K 31/454; A61K 31/573; A61K 47/68031; A61K 47/6849; A61P 35/00; A61K 2039/505

(57) ABSTRACT

A method to upregulate CD46 cell surface expression and combination therapies for various cancers employing an anti-CD46 antibody and an immunomodulatory imide drug (IMiD) or a Signal Transducer And Activator of Transcription 3 (STAT3) inhibitor or both are provided.

### CROSS-REFERENCE TO RELATED PATENT APPLICATIONS

The present patent application is a 371 U.S. National Phase application of International application No. PCT/US22/11500 filed Jan. 6, 2022; which claims benefit of priority to U.S. Provisional Patent Application No. 63/134,720, filed Jan. 7, 2021, which are incorporated by reference for all purposes.

### STATEMENT AS TO RIGHTS TO INVENTIONS MADE UNDER FEDERALLY SPONSORED RESEARCH AND DEVELOPMENT

This invention was made with government support under grants R01 CA118919 and R01 CA171315 awarded by The National Institutes of Health. The government has certain rights in the invention.

### SEQUENCE LISTING

The instant application contains a Sequence Listing which has been submitted electronically in ASCII format and is hereby incorporated by reference in its entirety. Said ASCII copy, created on Feb. 14, 2022, is named 081906-1290307-241610PC_SL.txt and is 111,375 bytes in size.

### BACKGROUND OF THE INVENTION

Due to ease of accessibility, tumor cell surface antigens are valuable targets for therapeutic development. The epitope space at the cell surface is highly complex. Relevant antigens may include glycosylated proteins and other post-translationally modified products that may not be readily predicted from studies of genomic copy number or mRNA expression levels (Liu et al. (2004) Cancer Res. 64: 704-710; Kobata and Amano (2005) Immunol. Cell Biol. 83: 429-439; Birkle et al. (2003) Biochimie (Paris) 85: 455-463; Hakomori (2001) Adv. Exp. Med. Biol. 491: 369-402; Hanisch, F. G. (2001) 0-Glycosylation of the mucin type. Biol. Chem. 382, 143-1 49; Ugorski and Laskowska (2002) Acta Biochim. Pol. 49: 303-311).

Identification of tumor cell surface epitopes allows the production of antibodies to achieve specific binding to neoplastic cells, an ability that can be utilized in applications such as induction of antibody-dependent cell cytotoxicity (see, e.g., Clynes et al. (2000) Nat. Med. 6: 443-446), or inhibition of signaling pathways involved in tumor cell migration, growth, and survival (see, e.g., McWhirter et al. (2006) Proc. Natl. Acad. Sci., USA, 103: 1041-1046; Fuh et al. (2006) J. Biol. Chem. 281: 6625-6631). In addition, antibodies targeting internalizing tumor epitopes can be exploited to achieve efficient and specific intracellular delivery of cytotoxins, cytostatic agents, chemotherapeutic drugs and/or other tumor-modulating agents (see, e.g., Liu et al. (2004) Cancer Res. 64: 704-710; Nielsen et al. (2002) Biochim. Biophys. Acta 1591: 109-118; Pirollo et al. (2006) Hum. Gene Ther. 17: 117-124; Song et al. (2005) Nat. Biotechnol. 23:709-717; Liu et al. (2002) J. Mol. Biol. 315: 1063-1073).

We have previously taken an unbiased affinity proteomic approach to map the tumor cell surface epitope space. We used a multi-billion member human antibody phage display library as a source of random shape repertoire and selected it on patient specimens and live tumor cells following counter-selection on normal tissues/cells (Ruan W, Sassoon A, An F, Simko J P, Liu B. Mol Cell Proteomics. 2006 Dec. 5(12):2364-73), and identified a panel of novel anti-CD46 human monoclonal antibodies that bind to a tumor selective epitope (Sherbenou D W, Aftab B T, Su Y, Behrens C R, Wiita A, Logan A C, Acosta-Alvear D, Hann B C, Walter P, Shuman M A, Wu X, Atkinson J P, Wolf J L, Martin T G, Liu B. J Clin Invest. 2016 Dec. 1; 126(12):4640-4653; Su Y, Liu Y, Behrens C R, Bidlingmaier S, Lee N K, Aggarwal R, Sherbenou D W, Burlingame A L, Hann B C, Simko J P, Premasekharan G, Paris P L, Shuman M A, Seo Y, Small E J, Liu B. JCI Insight. 2018 Sep. 6; 3(17):e12149.). A variety of anti-CD46 antibodies are known, including but not limited to those described in U.S. Pat. Nos. 9,593,162; 9,567,402 and 10,533,056.

### BRIEF SUMMARY OF THE INVENTION

In some embodiments, a method of treating multiple myeloma, lymphoma (including but not limited to Hodgkin's lymphoma), acute myeloid leukemia (AML), prostate cancer or metastatic renal cell carcinoma (mRCC) in a human is provided. In some embodiments, the method comprises administering to the human: an antibody that specifically binds to CD46, wherein the antibody is linked to a cytotoxic effector; and an agent that increases CD46 expression in cancer cells, wherein the agent is an immunomodulatory imide drug (IMid), optionally in combination with a glucocorticoid receptor agonist or modulator (SEGRAM), wherein administration of the antibody and the agent kills more cancer cells than administration of the antibody alone. In some embodiments, the agent is selected from the group consisting of pomalidamide, lenolidamide, thalidomide, iberdomide, and apremilast. In some embodiment, the administering comprises administering the agent without the antibody for a time sufficient to induce increased expression of CD46 in cancer cells followed by administering the antibody in an amount sufficient to kill myeloma cells in the human. In some embodiments, administering the antibody further comprises administering an IMid, a SEGRAM, or both with the antibody. In some embodiments, the SEGRAM is selected from the group consisting of dexamethasone, prednisone, cortisol, mapracorat, fosdagrocorat (PF-04171327), and dagrocorat. In some embodiments, the time comprises 1-30 days (e.g., 2-20, or 3-15, 5-10 days) before administering the antibody.

In some embodiments, the antibody comprises heavy chain CDRs 1, 2 and 3 and light chain CDRs 1, 2, and 3 of any one of YS5, YS5F, YS5vlD, SB1HGNY, YS12, 3G7RY (aka 3G8), YS6, YS1, YS3, YS4, YS8, YS7, YS9, YS10, YS11, 3G7HY, 3G7NY, 3G7, SB2, 2C8, or UA8kappa. In some embodiments, the antibody comprises a heavy chain (HC) variable region that comprises three complementarity determining regions (CDRs): HC CDR1, HC CDR2 and HC CDR3 and a light chain (LC) variable region that comprises three CDRs: LC CDR1, LC CDR2, and LC CDR3, wherein said HC CDR1, HC CDR2, HC CDR3 comprise an amino acid sequence of SEQ ID NO: 80, SEQ ID NO: 81, and SEQ ID NO: 82, respectively, and said LC CDR1, LC CDR2, and LC CDR3 comprise an amino acid sequence of SEQ ID NO: 83, SEQ ID NO: 84, and SEQ ID NO: 85, respectively.

In some embodiments, the cytotoxic effector is a chemotherapeutic agent. In some embodiments, the cytotoxic effector is a microtubule inhibitor, a DNA-damaging agent, or a polymerase inhibitor. In some embodiments, the cytotoxic effector is selected from the group consisting of an auristatin, Dolastatin-10, synthetic derivatives of the natural product Dolastatin-10, and maytansine or a maytansine derivative. In some embodiments, the cytotoxic effector is selected from the group consisting Monomethylauristatin F (MMAF), Auristatin E (AE), Monomethylauristatin E (MMAE), vcMMAE, and vcMMAF.

In some embodiments, the antibody comprises a heavy chain (HC) variable region that comprises three complementarity determining regions (CDRs): HC CDR1, HC CDR2 and HC CDR3 and a light chain (LC) variable region that comprises three CDRs: LC CDR1, LC CDR2, and LC CDR3, wherein said HC CDR1, HC CDR2, HC CDR3 comprise an amino acid sequence of SEQ ID NO: 80, SEQ ID NO: 81, and SEQ ID NO: 82, respectively, and said LC CDR1, LC CDR2, and LC CDR3 comprise an amino acid sequence of SEQ ID NO: 83, SEQ ID NO: 84, and SEQ ID NO: 85, respectively; and monomethylauristatin E (MMAE) that is conjugated to said antibody via a maleimidocaproyl-valine-citrulline-para-amino benzyloxycarbonyl (mc-vc-PAB) linker. In some embodiments, the HC comprises SEQ ID NO:86 and the LC comprises SEQ ID NO:87.

Also provided is a pharmaceutical composition comprising an anti-CD46 antibody conjugated to a cytotoxic effector; and an agent that is an immunomodulatory imide drug (IMid) that increases CD46 expression in cancer cells), optionally in combination with a glucocorticoid receptor agonist or modulator (SEGRAM). In some embodiments, the agent is selected from the group consisting of pomalidamide, lenolidamide, thalidomide, iberdomide, and apremilast. In some embodiments, the SEGRAM is selected from the group consisting of dexamethasone, prednisone, cortisol, mapracorat, fosdagrocorat (PF-04171327), and dagrocorat.

In some embodiments, the antibody comprises heavy chain CDRs 1, 2 and 3 and light chain CDRs 1, 2, and 3 of any one of YS5, YS5F, YS5vlD, SB1HGNY, YS12, 3G7RY (aka 3G8), YS6, YS1, YS3, YS4, YS8, YS7, YS9, YS10, YS11, 3G7HY, 3G7NY, 3G7, SB2, 2C8, or UA8kappa. In some embodiments, the antibody comprises a heavy chain (HC) variable region that comprises three complementarity determining regions (CDRs): HC CDR1, HC CDR2 and HC CDR3 and a light chain (LC) variable region that comprises three CDRs: LC CDR1, LC CDR2, and LC CDR3, wherein said HC CDR1, HC CDR2, HC CDR3 comprise an amino acid sequence of SEQ ID NO: 80, SEQ ID NO: 81, and SEQ ID NO: 82, respectively, and said LC CDR1, LC CDR2, and LC CDR3 comprise an amino acid sequence of SEQ ID NO: 83, SEQ ID NO: 84, and SEQ ID NO: 85, respectively.

In some embodiments, the cytotoxic effector is a chemotherapeutic agent. In some embodiments, the cytotoxic effector is a microtubule inhibitor, a DNA-damaging agent, or a polymerase inhibitor. In some embodiments, the cytotoxic effector is selected from the group consisting of an auristatin, Dolastatin-10, synthetic derivatives of the natural product Dolastatin-10, and maytansine or a maytansine derivative. In some embodiments, the cytotoxic effector is selected from the group consisting Monomethylauristatin F (MMAF), Auristatin E (AE), Monomethylauristatin E (MMAE), vcMMAE, and vcMMAF.

In some embodiments, the antibody comprises a heavy chain (HC) variable region that comprises three complementarity determining regions (CDRs): HC CDR1, HC CDR2 and HC CDR3 and a light chain (LC) variable region that comprises three CDRs: LC CDR1, LC CDR2, and LC CDR3, wherein said HC CDR1, HC CDR2, HC CDR3 comprise an amino acid sequence of SEQ ID NO: 80, SEQ ID NO: 81, and SEQ ID NO: 82, respectively, and said LC CDR1, LC CDR2, and LC CDR3 comprise an amino acid sequence of SEQ ID NO: 83, SEQ ID NO: 84, and SEQ ID NO: 85, respectively; and monomethylauristatin E (MMAE) that is conjugated to said antibody via a maleimidocaproyl-valine-citrulline-para-amino benzyloxycarbonyl (mc-vc-PAB) linker. In some embodiments, the HC comprises SEQ ID NO:86 and the LC comprises SEQ ID NO:87.

Also provided is a method of treating multiple myeloma, lymphoma, acute myeloid leukemia (AML), prostate cancer or metastatic renal cell carcinoma (mRCC) in a human, comprising
administering to the human:
an antibody that specifically binds to CD46, wherein the antibody is linked to a cytotoxic effector; and
an agent that increases CD46 expression in cancer cells, wherein the agent is a Signal Transducer And Activator of Transcription 3 (STAT3) inhibitor, optionally in combination with an immunomodulatory imide drug (IMiD) or a glucocorticoid receptor agonist or modulator (SEGRAM) or both, wherein administration of the antibody and the agent kills more cancer cells than administration of the antibody alone.

In some embodiments, the agent is selected from the group consisting of N-(1′, 2-Dihydroxy-1,2′-binaphthalen-4′-yl)-4-methoxybenzenesulfonamide (C188-9), STAT3 Inhibitor V, 6-Nitrobenzo[b]thiophene 1,1-dioxide (Stattic), (1E,6E)-1,7-Bis(4-hydroxy-3-methoxyphenyl)-1,6-heptadiene-3,5-dione (curcumin), N-Hexyl-2-(1-naphthalenyl)-5-[[4-(phosphonooxy)phenyl]methyl]-4-oxazolecarboxamide (S3I-M2001), 8-hydroxy-3-methyl-3,4-dihydrotetraphene-1,7,12(2H)-trione (STA-21), 2-Hydroxy-4-[[2-[[(4-methylphenyl)sulfonyl]oxy]acetyl]amino]benzoic acid (S3I-201), Cepharanthine, Cucurbitacin I, Cucumis sativus L, Niclosamide, Cryptotanshinone, SD 1008, Stat3 Inhibitor III, WP1066, Nifuroxazide, Stat3 Inhibitor VI, S3I-201, STA-21, Kahweol, STAT3 Inhibitor IX, Cpd188; STAT3 Inhibitor VI, 531-201; STAT3 Inhibitor VII Ethyl-1-(4-cyano-2,3,5,6-tetrafluorophenyl)-6,7,8-trifluoro-4-oxo-1,4-dih-ydroquinoline-3-carboxylate; STAT3 Inhibitor VIII, 5,15-DPP, STAT3 Inhibitor X, HJB; STAT3 Inhibitor XII, SPI; STAT3 Inhibitor XI, STX-0119; STAT3 Inhibitor XIV, LLL12; FLLL32; FLLL62; Napabucasin (BB1I608); DSP-0337 (prodrug of napabucasin); OPB-51602; OPB-31121; OPB-111077; Pyrimethamine; WP1066 and derivatives or analogues thereof.

In some embodiments, the administering comprises administering the agent without the antibody for a time sufficient to induce increased expression of CD46 in cancer cells followed by administering the antibody in an amount sufficient to kill myeloma cells in the human. In some embodiments, administering the antibody further comprises administering the agent, a SEGRAM, or both with the antibody.

In some embodiments, the SEGRAM is selected from the group consisting of dexamethasone, prednisone, cortisol, mapracorat, fosdagrocorat (PF-04171327), and dagrocorat.

In some embodiments, the time comprises 1-30 days (e.g., 2-20, or 3-15, 5-10 days) before administering the antibody.

In some embodiments, the antibody comprises heavy chain CDRs 1, 2 and 3 and light chain CDRs 1, 2, and 3 of any one of YS5, YS5F, YS5vlD, SB1HGNY, YS12, 3G7RY (aka 3G8), YS6, YS1, YS3, YS4, YS8, YS7, YS9, YS10, YS11, 3G7HY, 3G7NY, 3G7, SB2, 2C8, or UA8kappa.

In some embodiments, the antibody comprises a heavy chain (HC) variable region that comprises three complementarity determining regions (CDRs): HC CDR1, HC CDR2 and HC CDR3 and a light chain (LC) variable region that comprises three CDRs: LC CDR1, LC CDR2, and LC CDR3, wherein said HC CDR1, HC CDR2, HC CDR3 comprise an amino acid sequence of SEQ ID NO: 80, SEQ ID NO: 81, and SEQ ID NO: 82, respectively, and said LC CDR1, LC CDR2, and LC CDR3 comprise an amino acid sequence of SEQ ID NO: 83, SEQ ID NO: 84, and SEQ ID NO: 85, respectively.

In some embodiments, the cytotoxic effector is a chemotherapeutic agent. In some embodiments, the cytotoxic effector is a microtubule inhibitor, a DNA-damaging agent, or a polymerase inhibitor. In some embodiments, the cytotoxic effector is selected from the group consisting of an auristatin, Dolastatin-10, synthetic derivatives of the natural product Dolastatin-10, and maytansine or a maytansine derivative. In some embodiments, the cytotoxic effector is selected from the group consisting Monomethylauristatin F (MMAF), Auristatin E (AE), Monomethylauristatin E (MMAE), vcMMAE, and vcMMAF.

In some embodiments, the antibody comprises a heavy chain (HC) variable region that comprises three complementarity determining regions (CDRs): HC CDR1, HC CDR2 and HC CDR3 and a light chain (LC) variable region that comprises three CDRs: LC CDR1, LC CDR2, and LC CDR3, wherein said HC CDR1, HC CDR2, HC CDR3 comprise an amino acid sequence of SEQ ID NO: 80, SEQ ID NO: 81, and SEQ ID NO: 82, respectively, and said LC CDR1, LC CDR2, and LC CDR3 comprise an amino acid sequence of SEQ ID NO: 83, SEQ ID NO: 84, and SEQ ID NO: 85, respectively; and (b) monomethylauristatin E (MMAE) that is conjugated to said antibody via a maleimidocaproyl-valine-citrulline-para-amino benzyloxycarbonyl (mc-vc-PAB) linker. In some embodiments, the HC comprises SEQ ID NO:86 and the LC comprises SEQ ID NO:87.

Also provided is a pharmaceutical composition comprising an anti-CD46 antibody conjugated to a cytotoxic effector; and an agent that is a Signal Transducer And Activator of Transcription 3 (STAT3) inhibitor at a concentration sufficient to increase CD46 expression in cancer cells, optionally in combination with a glucocorticoid receptor agonist or modulator (SEGRAM).

In some embodiments, the agent is selected from the group consisting of N-(1′, 2-Dihydroxy-1,2′-binaphthalen-4′-yl)-4-methoxybenzenesulfonamide (C188-9), STAT3 Inhibitor V, 6-Nitrobenzo[b]thiophene 1,1-dioxide (Stattic), (1E,6E)-1,7-Bis(4-hydroxy-3-methoxyphenyl)-1,6-heptadiene-3,5-dione (curcumin), N-Hexyl-2-(1-naphthalenyl)-5-[[4-(phosphonooxy)phenyl]methyl]-4-oxazolecarboxamide (S3I-M2001), 8-hydroxy-3-methyl-3,4-dihydrotetraphene-1,7,12(2H)-trione (STA-21), 2-Hydroxy-4-[[2-[[(4-methylphenyl)sulfonyl]oxy]acetyl]amino]benzoic acid (S3I-201), Cepharanthine, Cucurbitacin I, Cucumis sativus L, Niclosamide, Cryptotanshinone, SD 1008, Stat3 Inhibitor III, WP1066, Nifuroxazide, Stat3 Inhibitor VI, S31-201, STA-21, Kahweol, STAT3 Inhibitor IX, Cpd188; STAT3 Inhibitor VI, 531-201; STAT3 Inhibitor VII Ethyl-1-(4-cyano-2,3,5,6-tetrafluorophenyl)-6,7,8-trifluoro-4-oxo-1,4-dih-ydroquinoline-3-carboxylate; STAT3 Inhibitor VIII, 5,15-DPP, STAT3 Inhibitor X, HJB; STAT3 Inhibitor XII, SPI; STAT3 Inhibitor XI, STX-0119; STAT3 Inhibitor XIV, LLL12; FLLL32; FLLL62; Napabucasin (BBI608); DSP-0337 (prodrug of napabucasin); OPB-51602; OPB-31121; OPB-111077; Pyrimethamine; WP1066 and derivatives or analogues thereof.

In some embodiments, the SEGRAM is selected is selected from the group consisting of dexamethasone, prednisone, cortisol, mapracorat, fosdagrocorat (PF-04171327), and dagrocorat.

In some embodiments, the antibody comprises heavy chain CDRs 1, 2 and 3 and light chain CDRs 1, 2, and 3 of any one of YS5, YS5F, YS5vlD, SB1HGNY, YS12, 3G7RY (aka 3G8), YS6, YS1, YS3, YS4, YS8, YS7, YS9, YS10, YS11, 3G7HY, 3G7NY, 3G7, SB2, 2C8, or UA8kappa. In some embodiments, the antibody comprises a heavy chain (HC) variable region that comprises three complementarity determining regions (CDRs): HC CDR1, HC CDR2 and HC CDR3 and a light chain (LC) variable region that comprises three CDRs: LC CDR1, LC CDR2, and LC CDR3, wherein said HC CDR1, HC CDR2, HC CDR3 comprise an amino acid sequence of SEQ ID NO: 80, SEQ ID NO: 81, and SEQ ID NO: 82, respectively, and said LC CDR1, LC CDR2, and LC CDR3 comprise an amino acid sequence of SEQ ID NO: 83, SEQ ID NO: 84, and SEQ ID NO: 85, respectively.

In some embodiments, the cytotoxic effector is a chemotherapeutic agent. In some embodiments, the cytotoxic effector is a microtubule inhibitor, a DNA-damaging agent, or a polymerase inhibitor. In some embodiments, the cytotoxic effector is selected from the group consisting of an auristatin, Dolastatin-10, synthetic derivatives of the natural product Dolastatin-10, and maytansine or a maytansine derivative. In some embodiments, the cytotoxic effector is selected from the group consisting Monomethylauristatin F (MMAF), Auristatin E (AE), Monomethylauristatin E (MMAE), vcMMAE, and vcMMAF.

In some embodiments, the antibody comprises (a) a heavy chain (HC) variable region that comprises three complementarity determining regions (CDRs): HC CDR1, HC CDR2 and HC CDR3 and a light chain (LC) variable region that comprises three CDRs: LC CDR1, LC CDR2, and LC CDR3, wherein said HC CDR1, HC CDR2, HC CDR3 comprise an amino acid sequence of SEQ ID NO: 80, SEQ ID NO: 81, and SEQ ID NO: 82, respectively, and said LC CDR1, LC CDR2, and LC CDR3 comprise an amino acid sequence of SEQ ID NO: 83, SEQ ID NO: 84, and SEQ ID NO: 85, respectively; and (b) monomethylauristatin E (MMAE) that is conjugated to said antibody via a maleimidocaproyl-valine-citrulline-para-amino benzyloxycarbonyl (mc-vc-PAB) linker.

In some embodiments, the HC comprises SEQ ID NO:86 and the LC comprises SEQ ID NO:87.

### Definitions

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed. The section headings used herein are for organizational purposes only and are not to be construed as limiting the subject matter described. In this application, the use of the singular includes the plural unless specifically stated otherwise. It is noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

The term “immunomodulatory imide drug (IMiD)” as used herein refers to a class of drugs containing an imide group. Without being bound by theory, it is believed that IMiDs are useful in the treatment of cancers due to immunomodulatory, antiangiogenic, and antineoplastic properties and as described herein, IMiDs induce CD46 expression in cancer cells, rendering the cancer cells more susceptible to drugs (e.g., antibodies) that target CD46. The IMiD class of drugs includes, but is not limited to, thalidomide and its analogs. The term “analog” as used herein is a compound having a structure similar to that of another one, but differing from it in respect of a certain component, e.g., the analog can differ in one or more atoms, functional groups, or substructures, which are replaced with other atoms, groups, or substructures. Various IMiDs are known to those skilled in the art, including, for example, thalidomide, lenalidomide, pomalidomide, apremilast, iberdomide and analogs thereof. See, e.g., US Patent Publication No. US 2020/0254093. Additional thalidomide analogs are described in, e.g., US Patent Publication No. US2020/0325102.

A “Stat3 inhibitor” inhibits one or more activity of human Stat3. STAT3 activity can include for example, STAT3 phosphorylation, STAT3 dimerization, STAT3 binding to a polynucleotide comprising a STAT3 binding site, STAT3 binding to genomic DNA, activation of a STAT3 responsive gene and STAT3 nuclear translocation. US Patent Publication No. 2017/0000884 describes, for example, a non-limiting list of Stat3 inhibitors and methods for measuring their activity.

As used herein, ranges and amounts can be expressed as “about” a particular value or range. About also includes the exact amount. Hence “about 5 μL” means “about 5 μL” and also “5 μL.” Generally, the term “about” includes an amount that would be expected to be within experimental error.

The terms “antibody” and “immunoglobulin” are used interchangeably herein and are used in the broadest sense and covers fully assembled antibodies, antibody fragments that can bind antigen, for example, Fab, F(ab′)2, Fv, single chain antibodies (scFv), diabodies, antibody chimeras, hybrid antibodies, bispecific antibodies, and the like. The recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

The terms “monoclonal antibody” and “mAb” are used interchangeably herein and refer to an antibody obtained from a substantially homogeneous population of antibodies, i.e., the individual antibodies of the population are identical except for possible naturally occurring mutations that may be present in minor amounts.

The term “hypervariable region,” as used herein, refers to the amino acid residues of an antibody that are responsible for antigen-binding. The hypervariable region comprises amino acid residues from a “complementarily determining region” or “CDR” (i.e., residues 24-34 (L1), 50-56 (L2), and 89-97 (L3) in the light-chain variable domain and 31-35 (H1), 50-65 (H2), and 95-102 (H3) in the heavy-chain variable domain; Kabat et al. (1991) Sequences of Proteins of Immunological Interest Fifth Edition, U.S. Department of Health and Human Services, NIH Publication No. 91-3242 (referred to herein as “Kabat et al”) and/or those residues from a “hypervariable loop” (i.e., residues 26-32 (L1), 50-52 (L2), and 91-96 (L3) in the light-chain variable domain and (H1), 53-55 (H2), and 96-101 (13) in the heavy chain variable domain; Chothia and Lesk, (1987) J. Mol. Biol., 196:901-917). “Framework” or “FR” residues are those variable domain residues other than the hypervariable region residues, as herein deemed.

In some instances, the CDRs of an antibody is determined according to (i) the Kabat numbering system Kabat et al. (1991) Sequences of Proteins of Immunological Interest Fifth Edition, U.S. Department of Health and Human Services, NIH Publication No. 91-3242; or (ii) the Chothia numbering scheme, which will be referred to herein as the “Chothia CDRs” (see, e.g., Chothia and Lesk, 1987, J. Mol. Biol., 196:901-917; Al-Lazikani et al., 1997, J. Mol. Biol., 273:927-948; Chothia et al., 1992, J. Mol. Biol., 227:799-817; Tramontano A et al., 1990, J. Mol. Biol. 215(1): 175-82; and U.S. Pat. No. 7,709,226); or (iii) the ImMunoGeneTics (IMGT) numbering system, for example, as described in Lefranc, M.-P., 1999, The Immunologist, 7: 132-136 and Lefranc, M.-P. et al, 1999, Nucleic Acids Res., 27:209-212 (“IMGT CDRs”); or (iv) MacCallum et al, 1996, J. Mol. Biol., 262:732-745. See also, e.g., Martin, A., “Protein Sequence and Structure Analysis of Antibody Variable Domains,” in Antibody Engineering, Kontermann and Diibel, eds., Chapter 31, pp. 422-439, Springer-Verlag, Berlin (2001).

With respect to the Kabat numbering system, CDRs within an antibody heavy chain molecule are typically present at amino acid positions 31 to 35, which optionally can include one or two additional amino acids, following 35 (referred to in the Kabat numbering scheme as 35 A and 35B) (CDR1), amino acid positions 50 to 65 (CDR2), and amino acid positions 95 to 102 (CDR3). Using the Kabat numbering system, CDRs within an antibody light chain molecule are typically present at amino acid positions 24 to 34 (CDR1), amino acid positions 50 to 56 (CDR2), and amino acid positions 89 to 97 (CDR3). As is well known to those of skill in the art, using the Kabat numbering system, the actual linear amino acid sequence of the antibody variable domain can contain fewer or additional amino acids due to a shortening or lengthening of a FR and/or CDR and, as such, an amino acid's Kabat number is not necessarily the same as its linear amino acid number.

As used herein, the term “antigen-binding site” refers to the part of the antigen binding molecule that specifically binds to an antigenic determinant. More particularly, the term “antigen binding site” refers the part of an antibody that comprises the area which specifically binds to and is complementary to part or all of an antigen. Where an antigen is large, an antigen binding molecule may only bind to a particular part of the antigen, which part is termed an epitope. An antigen-binding site may be provided by, for example, one or more variable domains (also called variable regions). Preferably, an antigen-binding site comprises an antibody light chain variable region (VL) and an antibody heavy chain variable region (VH).

By “specific binding” is meant that the binding is selective for the antigen and can be discriminated from unwanted or non-specific interactions. The ability of an antigen binding molecule to bind to a specific antigen can be measured either through an enzyme-linked immunosorbent assay (ELISA) or other techniques familiar to one of skill in the art, e.g. Surface Plasmon Resonance (SPR) technique (analyzed on a BIAcore instrument) (Liljeblad et al., Glyco J 17, 323-329 (2000)), and traditional binding assays (Heeley, Endocr Res 28, 217-229 (2002)). In one embodiment, the extent of binding of an antigen binding molecule to an unrelated protein is less than about 10% of the binding of the antigen binding molecule to the antigen as measured, e.g. by SPR. In certain embodiments, a molecule that binds to the antigen has a dissociation constant (Kd) of ≤1 μM, ≤100 nM, ≤10 nM, ≤1 nM, ≤0.1 nM, ≤0.01 nM, or ≤0.001 nM (e.g. 10-7 M or less, e.g. from 10-⁷M to 10-¹³ M, e.g. from 10-⁹ M to 10-¹³ M).

As noted above, depending on the amino acid sequence of the constant domain of their heavy chains, immunoglobulins can be assigned to different classes. There are five major classes of human immunoglobulins: IgA, IgD, IgE, IgG, IgM, and IgY, and several of these may be further divided into subclasses (isotypes), e.g., IgG1, IgG2, IgG3, IgG4, IgA1, and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known. Different isotypes have different effector functions. For example, human IgG1 and IgG3 isotypes have ADCC (antibody dependent cell-mediated cytotoxicity) activity. The light chains of antibodies (immunoglobulins) from any vertebrate species can be assigned to one of two clearly distinct types, called kappa (x) and lambda (k), based on the amino acid sequences of their constant domains.

The term “chimeric antibody,” as used herein refers to an antibody in which a portion of the heavy and/or light chain is derived from a particular source (e.g., protein) or species, while the remainder of the heavy and/or light chain is derived from a different source (e.g., protein) or species.

The term “recombinant human antibody,” as used herein, is intended to include all human antibodies that are prepared, expressed, created or isolated by recombinant means, such as antibodies isolated from a host cell such as a NSO or CHO cell or from an animal (e.g. a mouse) that is transgenic for human immunoglobulin genes or antibodies expressed using a recombinant expression vector transfected into a host cell. Such recombinant human antibodies have variable and constant regions in a rearranged form. In some cases, the recombinant human antibodies have been subjected to in vivo somatic hypermutation. Thus, the amino acid sequences of the VH and VL regions of the recombinant antibodies are sequences that, while derived from and related to human germ line VH and VL sequences, may not naturally exist within the human antibody germ line repertoire in vivo.

The term “valent” as used herein denotes the presence of a specified number of binding sites in an antigen binding molecule. As such, the terms “bivalent”, “tetravalent”, and “hexavalent” denote the presence of two binding sites, four binding sites, and six binding sites, respectively, in an antigen binding molecule. The bispecific antibodies according to the invention are at least “bivalent” and may be “trivalent” or “multivalent” (e.g. “tetravalent” or “hexavalent”). In a particular aspect, the antibodies of the present invention have two or more binding sites and are bispecific. That is, the antibodies may be bispecific even in cases where there are more than two binding sites (i.e. that the antibody is trivalent or multivalent). In particular, the invention relates to bispecific bivalent antibodies, having one binding site for each antigen they specifically bind to.

The term “monospecific” antibody as used herein denotes an antibody that has one or more binding sites each of which bind to the same epitope of the same antigen.

The terms “individual(s)”, “subject(s)” and “patient(s)” are used interchangeably herein and refer to any mammal. In some embodiments, the mammal is a human. In some embodiments, the mammal is a non-human. None of the terms require or are limited to situations characterized by the supervision (e.g. constant or intermittent) of a health care worker (e.g. a doctor, a registered nurse, a nurse practitioner, a physician's assistant, an orderly or a hospice worker).

The terms “cancer” and “tumor” are used interchangeably herein, encompass all types of oncogenic processes and/or cancerous growths. In embodiments, cancer includes primary tumors as well as metastatic tissues or malignantly transformed cells, tissues, or organs. In embodiments, cancer encompasses all histopathologies and stages, e.g., stages of invasiveness/severity, of a cancer. In embodiments, cancer includes relapsed and/or resistant cancer.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, the molecules described herein are used to delay development of a disease or to slow the progression of a disease.

### BRIEF DESCRIPTION OF THE DRAWINGS

FIG. 1: Immunomodulatory imide drugs (IMiDs) pre-treatment leads to CD46 upregulation in multiple myeloma. RPMI8226 cells were treated with IMiDs (Pomalidomide and Lenalidomide) for 3 and 10 days. CD46 expression was analyzed by FACS. CD38 was also measured as a reference. IMiDs-induced cell surface upregulation was observed only to selective antigens, e.g., CD46 but not CD38.

FIG. 2: Pomalidomide-induced upregulation persists following initial exposure. The left portion displays CD46 upregulation on RPMI8226 cells following 7-day exposure to varying concentrations of pomalidomide and lenalidomide. FACS was performed on day-7. The right portion displays CD46 upregulation on RPMI8226 cells following a transient 7-day exposure to varying concentrations of pomalidomide and lenalidomide and 7-day recovery period. FACS was performed on day-14.

FIG. 3: Pomalidomide exposure, followed by CD46 ADC as a single agent. RPMI8226 seeded at 5,000 per well was exposed to 3.3 μM, 5 μM, and 10 μM pomalidomide for 7 days, washed, and treated with CD46 ADC. Cell viability was measured 96 h post ADC treatment. EC50 changed little, but EC65 (i.e., 35% viability) changed over 100-fold (no pre-treatment vs. 10 μM pom pre-treatment).

FIG. 4: Lenalidomide exposure, followed by CD46 ADC as a single agent. RPMI8226 seeded at 5,000 per well was exposed to 3.3 μM, 5 μM, 10 μM lenalidomide for 7 days, washed, and treated with CD46 ADC. Cell viability was measured 96 h post ADC treatment. EC50 changed little, but EC65 (i.e., 35% viability) changed over 100-fold (no pre-treatment vs. 10 μM len pre-treatment).

FIG. 5: Exposure to pomalidomide (pom), followed by combination treatment with pom plus CD46 ADC. RPMI8226 seeded at 3,000 per well was exposed to 10 μM pomalidomide for 7 days, and treated with 10 μM pomalidomide plus CD46 ADC. Cell viability was measured 96 h post combination treatment. EC50=3.717 nM for CD46 ADC (single agent, no pretreatment with pom), 3.438 nM for pom+CD46 ADC combo (no pretreatment), and 1.297 nM for pom pretreatment followed by pom plus CD46 ADC combo.

FIG. 6: Exposure to pom and dex further increases cell surface CD46 expression. The multiple myeloma cell line RPMI8226 was incubated with 10 μM pomalidamide (pom) for 14 days, washed, and further incubated with 10 nM dexamethasone (dex) for 7 days. Cell surface CD46 expression was assessed by flow cytometry. MFI: median fluorescence intensity. Multiple group comparison was performed using One-way ANOVA. **p<0.01; ****p<0.0001.

FIG. 7: Pomalidomide (pom) lead-in plus pom/dexamethasone (dex)/CD46 ADC combination further enhances anti-tumor potency. RPMI8226 cells were exposed to 10 μM pomalidomide, followed by incubation with 0.2 nM CD46 ADC (CD46ADCMMAE), or 0.2 nM CD46 ADC+10 μM pom+10 nM dex (combo) for 4 days. Single treatments with 10 μM pom or 10 nM dex are also performed as controls. Cell viability was normalized and compared by One-Way ANOVA. Multiple comparison analysis was performed against the combo group. ***p<0.001; ****p<0.0001.

FIG. 8. Effects of C188-9 on multiple myeloma cells. RMPI8226 cells were incubated with varying concentration of C188-9 at 37° C. for 7 days and analyzed by flow cytometry for CD46 cell surface expression. MFI: median fluorescence intensity. One-way ANOVA, *P<0.05; ***P<0.001. C188-9 stimulates at low concentration but inhibits at high concentration CD46 expression in multiple myeloma cells.

FIG. 9. Effects of the glucocorticoid receptor inhibitor mifepristone on multiple myeloma cells. RPMI8226 cells were incubated with mifepristone at 37° C. for 7 days, and analyzed by flow cytometry for CD46 cell surface expression. MFI: median fluorescence intensity. One-way ANOVA, *P<0.05; ns: not significant. Mifepristone inhibits CD46 cell surface expression at high concentrations, further showing that the glucocorticoid signaling pathway is involved in CD46 regulation.

### DETAILED DESCRIPTION OF THE INVENTION

The methods and compositions described herein include usage of an anti-CD46 antibody in combination with an immunomodulatory imide drug (IMiD) or Signal Transducer And Activator of Transcription 3 (Stat3) inhibitor as described herein. This combination can result in significant improvement of the efficacy of the anti-CD46 antibody. Without intending to limit the scope of the invention it is believed that IMiDs and Stat3 inhibitors induce expression of CD46 in cancer cells thereby allowing for improved efficacy of the anti-CD46 antibody, which can be linked to an effector molecule such as a cytotoxin. For example, the inventors have discovered that pomalidomide or other IMiDs can initially be administered alone for a first period of time resulting in increased expression of CD46 in cancer cells followed by administration of the anti-CD46 antibody (optionally linked to an effector molecule such as a cytotoxin) for a second period. Further improvement in results can be observed by co-administration of IMiDs with the anti-CD46 antibody in the second period of time. A similar approach can be used in the context of STAT3 inhibitors and an anti-CD46 antibody.

In some embodiments, the anti-CD46 antibody is an internalizing antibody, meaning that the antibodies are internalized by tumor cells, for example via the macropinocytosis pathway. For example, the antibodies can be internalized by the tumor-selective macropinocytosis pathway, without the need of crosslinking, and localize to the lysosomes, which makes them well suited for use as antibody drug conjugates (ADCs) and other targeted therapeutics that utilize intracellular payload release. A large number of anti-CD46 antibodies are known, including but not limited to those described in U.S. Pat. Nos. 9,593,162; 9,567,402 and 10,533,056.

In some embodiments, the anti-CD46 specifically bind CD46, in particular domains 1 and/or 2, and are internalized by multiple myeloma cells (and other CD46 positive cancer cells, such as those described herein) in situ, e.g., when the cancer cell is in the tissue microenvironment. As indicated above, such antibodies are useful for targeting cancers when used alone, or when attached to an effector to form a “targeted effector”.

The antibodies designated herein as YS5, YS5F, YS5vlD, SB1HGNY, YS12, 3G7RY (aka 3G8), YS6, YS1, YS3, YS4, YS8, YS7, YS9, YS10, YS11, 3G7HY, 3G7NY, 3G7, SB2, 2C8, and UA8kappa (see, e.g., Table 1) are exemplary anti-CD46 antibodies. In certain embodiments, antibodies that comprise VL CDR1 and/or VL CDR2, and/or VL CDR3, and/or VH CDR1 and/or VH CDR2, and/or VH CDR3 of one or more of these antibodies are contemplated. In certain embodiments, antibodies that comprise the VH domain and/or the VL domain of one or more of these antibodies are contemplated. Also contemplated are antibodies that compete for binding at CD46 with one or more of as YS5, YS5F, YS5vlD, SB1HGNY, YS12, 3G7RY (aka 3G8), YS6, YS1, YS3, YS4, YS8, YS7, YS9, YS10, YS11, 3G7HY, 3G7NY, 3G7, SB2, 2C8, and/or UA8kappa.

The amino acid sequences of the VH and VL domains of YS5, YS5F, YS5vlD, SB1HGNY, YS12, 3G7RY (aka 3G8), YS6, YS1, YS3, YS4, YS8, YS7, YS9, YS10, YS11, 3G7HY, 3G7NY, 3G7, SB2, 2C8, and/or UA8kappa antibodies are shown in Table 1.

TABLE 1
Novel human anti-CD46 antibody sequences. YS5 and YS5F differ by one
amino acid in VH CDR1 (L vs. F). YS5 and YS5vlD have identical VH but one amino acid
difference in the VL CDR2 (N vs. D). 3G7HY, 3G7NY, 3G7RY (aka 3G8), and 3G7 have one
residue difference in VH CDR3, but entirely different VLs. YS6 and 3G7 have identical
VH but different VL.

| | VH | VL |
|---|---|---|
| YS5 | QVQLVQSGGGVVQPGRSLRLACAASGLTV | QSVLTQPPSVSGAPGQRVTISCTGSSSNIGA |
| | NNYAMHWVRQAPGKGLEWVAVISYDGNNK | GYDVHWYQQLPGTAPKLLIYGNNNRPSGVPD |
| | YYADSVKGRFTISRDNSKNTLYLQMNSLR | RFSGSKSGTSASLAITGLQAEDEADYYCSSY |
| | AEDTAVYYCAKGGGYFDLWGRGTLVTVSS | TSGTWLFGGGTKLTVL |
| | (SEQ ID NO: 1) | (SEQ ID NO: 22) |
| YS5F | QVQLVQSGGGVVQPGRSLRLACAASGFTV | QSVLTQPPSVSGAPGQRVTISCTGSSSNIGA |
| | NNYAMHWVRQAPGKGLEWVAVISYDGNNK | GYDVHWYQQLPGTAPKLLIYGNNNRPSGVPD |
| | YYADSVKGRFTISRDNSKNTLYLQMNSLR | RFSGSKSGTSASLAITGLQAEDEADYYCSSY |
| | AEDTAVYYCAKGGGYFDLWGRGTLVTVSS | TSGTWLFGGGTKLTVL |
| | (SEQ ID NO: 2) | (SEQ ID NO: 23) |
| YS5VlD | QVQLVQSGGGVVQPGRSLRLACAASGFTV | QSVLTQPPSVSGAPGQRVTISCTGSSSNIGA |
| | NNYAMHWVRQAPGKGLEWVAVISYDGNNK | GYDVHWYQQLPGTAPKLLIYGDNNRPSGVPD |
| | YYADSVKGRFTISRDNSKNTLYLQMNSLR | RFSGSKSGTSASLAITGLQAEDEADYYCSSY |
| | AEDTAVYYCAKGGGYFDLWGRGTLVTVSS | TSGTWLFGGGTKLTVL |
| | (SEQ ID NO: 3) | (SEQ ID NO: 24) |
| SB1HG | QVQLQQSGGGVVQPGRSLRLSCAASGFTF | DIQMTQSPSFLSASVGDRVTITCRASQGISS |
| NY | SSYAMHWVRQAPGKGLEWVAFIRSDGSKK | YLAWYQQKPGKAPKLLIYAASTLQSGVPSSF |
| | YYADSVKGRFTISRDNSKNTLYLQMNSLR | SGSGSGTEFTLTISSLQPEDFATYYCQQLAS |
| | AEDTAVYYCARHGNYFDSWGQGTLVTVSS | YPLTFGGGTKVDIK |
| | (SEQ ID NO: 4) | (SEQ ID NO: 25) |
| YS12 | QVQLVESGGGVVQPGRSLRLSCAASGFTF | SSELTQDPAVSVALGQTVRITCQGDSLRSYY |
| | STYGMHWVRQAPGKGLEWLSFISYDGDEK | VSWFQQKPGQAPVFVMYGQNNRPSGISERFS |
| | YYADSVKGRFTISRDNSKNTLYLQMNSLR | GSSSGNTASLIITGAQAEDEADYYCHSRDSS |
| | AEDTAVYWCAKASGYGMGILDYWGQGTLV | GTHLRVFGGGTKLTVL |
| | TVSS | (SEQ ID NO: 26) |
| | (SEQ ID NO: 5) | |
| 3G7RY | EVQLVESGGGLVQPGGSLRLSCAASGFTF | QSALTQPPSASATPGQRVTISCSGRTSNIGS |
| aka | SDYYMSWIRQAPGKGLEWVSYISSSGSTI | NHVYWYQQLPGTAPKLLIYRNNQRPSGVPDR |
| 3G8 | YYADSVKGRFTI SRDNSKNTLYLQMNSLR | FSGSKSGTSASLAISGLRSEDEADYYCATWD |
| | AEDTAVYYCARDYGRIAAAGRRYWGQGTL | DSLSGEVFGGGTKLTVL |
| | VTVSS | (SEQ ID NO: 27) |
| | (SEQ ID NO: 6) | |
| YS6 | QVQLQESGGGVVRPGGSLRLSCAASGFTF | SSELTQDPAVSVALGQTVRITCQGDSLRSYY |
| | SDYYMSWIRQAPGKGLEWVSYISSSGSTI | ASWYQQKPGQAPVLVIYGKNNRPSGIPDRES |
| | YYADSVKGRFTISRDNSKNTLYLQMNSLR | GSSSGNTASLTITGAQAEDEADYYCNSRDSS |
| | AEDTAVYYCARDYGRIAAAGRHYWGQGTL | GTHLEVFGGGTKVTVL |
| | VTVSS | (SEQ ID NO: 28) |
| | (SEQ ID NO: 7) | |
| YS1 | EVQLVESGGGLVQPGGSLRLSCAASGFTF | SSELTQDPAVSVALGQTVRITCQGDTLSTYY |
| | SDYYMSWIRQAPGKGLEWVSYISSSGSTI | ANWYQQKPGQAPVLVIYGKNNRPSGIPDRES |
| | YYADSVKGRFTISRDNSKNTLYLQMNSLR | GSSSGNTASLTITGAQAEDEADYYCHSRDIS |
| | AEDTAVYYCARDYGRIAAAGRHYWGQGTL | GNYLFASGTKLTVL |
| | VTVSS | (SEQ ID NO: 29) |
| | (SEQ ID NO: 8) | |
| YS3 | QVQLQESGGGLVQPGGSLRLSCAASGFTF | QSVLTQPPSASGTPGQRVTISCSGSSSNIGS |
| | SSYWMSWVRQAPGKGLEWVADIKQDGSEK | NTVNWSRQLPGTAPKLLIYSNNQRPSGVPDR |
| | YYVDSVKGRFTI SGDNAKNSLYLQMNSLR | FSGSKSGTSASLAISGLQSEDEADYYCAAWD |
| | AEDTAVYYCAKDVGSTAINYVRAYTWFDP | DSLNVYVFGTGTKVTVL |
| | WGQGTLVTVSS | (SEQ ID NO: 30) |
| | (SEQ ID NO: 9) | |
| YS4 | QVQLQESGGGLVQPGGSLRLSCAASGFTF | KIVLTQSPSSLSASVGDTVTIACRASRDIRN |
| | SNYAMSWVRQAPGKGLEWVSTISGSGSST | DLAWYQQKPGKAPKLLIYGASSLQSGVPSRF |
| | FYVDSVKGRFTISRDNSKNTLYLQMNSLR | SGSGSGTEFILTISSLQPEDFATYYCHRINS |
| | AEDTAVYYCAQGLYSSGWANWFDPRGQGT | YPLTFGGGTKVDIK |
| | LVTVSS | (SEQ ID NO: 31) |
| | (SEQ ID NO: 10) | |
| YS8 | QVQLQESGGGVVQPGRSLRLSCAASGFTF | NFMLTQPASLSGSPGQSITISCTGTSSDVGG |
| | SSYGMHWVRQAPGKGLEWVAVISYDGSNK | YNYVSWYQQHPGYAPKLMIYDVSNRPSGVSN |
| | YYADSVKGRFTISRDNSKNTLYLQMNSLR | RFSGSKSGNTASLTISGLQAEDEADYYCSSY |
| | AEDTAVYYCAKVMGLAAAGLDAFDIWGQG | TSSSTPWVFGGGTKLTVL |
| | TTVTVSS | (SEQ ID NO: 32) |
| | (SEQ ID NO: 11) | |
| YS7 | QVQLVQSGGGVVQPGRSLRLSCAASGFTF | SYVLTQDPAVSVALGQTVRITCQGDSLRSYY |
| | SSYAMHWVRQAPGKGLEWVAVISYDGSNK | ASWYQQKPGQAPVLVIYGKNNRPSGIPDRES |
| | YYADSVKGRFTISRDTSTNTLYLQMNSLR | GSSSGNTASLTITGAQAEDEADYYCNSRDSS |
| | ADDTAVYYCGRESSGSPGVWGQGTTVTVS | GNQFGGGTKLTVL |
| | S | (SEQ ID NO: 33) |
| | (SEQ ID NO: 12) | |
| YS9 | QVQLVESGGGLIQPGGSLRLSCAASGFTV | SSELTQDPAVSVALGQTVRITCQGDSLRTYY |
| | SSNYMSWVRQAPGKGLEWVSVIYTDGSTY | ASWYQQRPGQAPILVLYGKNNRPSGIPDRES |
| | YADSVKGRFTISRDNSKNTLYLQMNSLRA | GSSSGNTASLTITGAQAEDEADYYCNSRDSS |
| | EDTAIYYCARDRGTSGYDWAWFDLWGQGT | GNHVVFGGGTKLTVL |
| | LVTVSS | (SEQ ID NO: 34) |
| | (SEQ ID NO: 13) | |
| YS10 | QVQLQESGGGLVQPGGSLRLSCAASGFTF | QSVLTQPASVSGSPGQSITISCTGTGSDVGS |
| | SSYAMSWVRQAPGKGLEWVSAISGSGGST | YNYVSWYQQNPGKAPKLMIYEVSNRPSGVSN |
| | YYADSVKGRFTISRDNSKNTLYMQMNSLR | RFSGSKSGNTASLTISGLQAEDEADYYCSSY |
| | AEDTAVYYCAKDRYYYGSGKDAFDIWGRG | TTSSTLVFGGGTKVTVL |
| | TMVTVSS | (SEQ ID NO: 35) |
| | (SEQ ID NO: 14) | |
| YS11 | QVQLVESGGGLVQPGGSLGLSCAASGFTF | SELTQDPAVSVALGQTVRITCQGDSLRSYYA |
| | SNYWMSWVRQAPGKGLEWVANVRQDGGQK | SWYQQKPGQAPVLVIYGENSRPSGIPDRESG |
| | YYVDSVKGRFTISRDNAKNSLYLQMNSLR | SSSGNTASLTITGAQAEDEADYYCNSWDSSG |
| | TEDTAVYFCVSQRNSGEHDYWGQGTLVTV | NHVVFGGGTKLTVL |
| | SS | (SEQ ID NO: 36) |
| | (SEQ ID NO: 15) | |
| 3G7HY | EVQLVESGGGLVQPGGSLRLSCAASGFTF | AIRMTQSPSSLSASVGDRVTITCRASQSISS |
| | SDYYMSWIRQAPGKGLEWVSYISSSGSTI | YLNWYQQKPGKAPKLLIYAASSLQSGVPSRE |
| | YYADSVKGRFTISRDNSKNTLYLQMNSLR | SGSGSGTDFTLTISSLQPEDFATYYCQQSYS |
| | AEDTAVYYCARDYGRIAAAGRHYWGQGTL | TPRTFGQGTKLEIK |
| | VTVSS | (SEQ ID NO: 37) |
| | (SEQ ID NO: 16) | |
| 3G7NY | EVQLVESGGGLVQPGGSLRLSCAASGFTF | DIVMTQSPLSLPVTPGEPASISCRSSQSLLH |
| | SDYYMSWIRQAPGKGLEWVSYISSSGSTI | SNGYDYLDWYLQKPGQSPQLLIYLGSNRASG |
| | YYADSVKGRFTISRDNSKNTLYLQMNSLR | VPDRFSGSGSGTDFTLKISRVETEDVGIYYC |
| | AEDTAVYYCARDYGRIAAAGRNYWGQGTL | MQGLQTPSFGQGTKLEIK |
| | VTVSS | (SEQ ID NO: 38) |
| | (SEQ ID NO: 17) | |
| 3G7 | QVQLQESGGGVVRPGGSLRLSCAASGFTF | SSELTQDPAVSVALGQTVRITCQGDSLRSYY |
| | SDYYMSWIRQAPGKGLEWVSYISSSGSTI | ASWYQQKPGQAPVPVIYGKNNRPSGIPDRES |
| | YYADSVKGRFTISRDNSKNTLYLQMNSLR | GSSSGNTASLTITGAQAEDEADYYCNSRDSS |
| | AEDTAVYYCARDYGRIAAAGRHYWGQGTL | STHRGVFGGGTKLTVL |
| | VTVSS | (SEQ ID NO: 39) |
| | (SEQ ID NO: 18) | |
| SB2 | EVQLVESGGGLVKPGGSLRLSCAASGFTF | DIQLTQSPSSLSASVGDRVTITCRASRSIST |
| | SDYYMSWIRQAPGKGLEWVSYISSSGSSI | YLSWYQQKPGKAPKLLIYDASRLQNGVPSRF |
| | YYADSVKGRFTISRDNAKNSLYLQMNSLK | SGSGSDTDFTLTISSLQPEDFATYFCQQSYN |
| | AEDTAVYYCARDITDVVGVSFDYWGQGTL | PPWTFGQGTKLEIK |
| | VTVSS | (SEQ ID NO: 40) |
| | (SEQ ID NO: 19) | |
| 2C8 | EVQLVESGGGVVQPGRSLRLSCAASGFTF | QSALTQPASVSGSPGQSITISCTGTSSDVGG |
| | SSYGMHWVRQAPGKGLEWVAVISYDGSNK | YNYVSWYQQHPGKAPKLMIYDVSNRPSGVSN |
| | YYADSVKGRFTISRDNSKNTLYLQMNSLR | RFSGSKSGNTASLTISGLQAEDEAYYYCSSY |
| | AEDTAEYYCAKVMGLAAAGLDAFDIWGQG | TSSSDPWVFGGGTQLTVL |
| | TLVTVSS | (SEQ ID NO: 41) |
| | (SEQ ID NO: 20) | |
| UA8kappa | EVQLVESGGGVVQPGRSLRLSCAASGFTF | NIQMTQSPSSLSASVGDRVTITCRAGQPIST |
| | SSFGMHWVRRAPGKGLEWVAVISYDGSNQ | YVNWYQHKPGKAPKLLIYGASNLQSGVPSRF |
| | YYADSVKGRFTISRDNSKNTLYLQMNSLR | SGGGSATDFTLTISSLQPEDFATYYCQQSYS |
| | AEDTAVYYCGSRPGGGYASGSTVAYWGQG | SLLTFGDGTKVEIK |
| | TLVTVSS | (SEQ ID NO: 42) |
| | (SEQ ID NO: 21) | |

In various embodiments the antibodies comprise the three VH CDRs and/or the three VL CDRs of antibodies 3051.1, G12FC3, M6c42b, 4F3YW, M40pr146, UA20, UA8, 585II41, 585II41.1, 585II56, 3076, 3051, M49R, RCI-14, II79_4, II79_3, T5II-4B.1, T5II-4B.2, RCI-11, RCI-20, CI-11A, CI-14A, or S95-2 that are described in PCT/US2008/076704 (WO 2009/039192) or the mPA7 antibody. The amino acid sequences of the VH and VL chains of these antibodies and the CDRs comprising these domains are shown in PCT/US2008/076704 and the amino acid sequences of these domains are reproduced below in Table 2.

TABLE 2
Additional antibodies. The sequence shown below are scFv antibodies (the
VL and VH regions are joined by a (Gly₄Ser)₃ (SEQ ID NO: 43) linker,
however it will be recognized that other antibody forms comprising the
CDRs (or the VH and/or VL domains) are possible.

| | | SEQ ID |
|---|---|---|
| Clone | Amino Acid Sequence | No |
| 3051.1 | QVQLQESGGGLVKPGGPLRLSCAASGFTFSSYGMYWVRQAPGKGLEWV | 44 |
| | STLSRSGSGTYYADSVKGRFTISRDNSKNTLYLQMNSLRAEDTAVYYC | |
| | ASIAVAGNYFDYWGQGTLVTVSSGGGGSGGGGSGGGGSSYVLTQDPAV | |
| | SVALGQTVRITCQGDSLRSYYASWYQERPGQAPLLVIYGKNNRPSGIP | |
| | DRFSGSNSGSTATLTISRVEAGDEGDYYCQVWDSINEQVVFGGGTKVT | |
| | VL | |
| G12FC3 | QVQLVQSGGGVVQPGRSLRLSCAATGIPFSGSGMHWVRQAPGKGLEWV | 45 |
| | TMIWYDGSNKFYADSVKGRFTISRDNSKNTLYLQMDSLRAEDTAVYFC | |
| | ARDKGVRSMDVWGLGTTVTVSSGGGGSGGGGSGGGGSNFMLTQPPSVS | |
| | VAPGQTAKITCDGYSIRTKSVHWYQQKPGQAPVVVVHDDSDRPSGIPE | |
| | RFSGSNSGTTATLTISRVEAGDEADYYCQAWDSISEEVVFGGGTKLTV | |
| | L | |
| M6c42b | QVQLQESGGGLVQPGGSLRLSCSASGFTFGTYAMRWVRQTSGKGLEWV | 46 |
| | SGIGVSGDAYYTDSVRGRFTISRDNSKNTLYLQMNTLRAEDTATYYCT | |
| | RKSSTTSNDYWGRGTLVTVSSGGGGSGGGGSGGGGSSYVLTQDPAVSV | |
| | ALGQTVRITCQGDNIGSKSVHWYQQKPGQAPVLVVYDDSDRPSGIPER | |
| | FSGSNSGTTATLTISSVEAGDEADYYCQAWDSISEHVIFGGGTKVTVL | |
| 4F3YW | QVQLQESGGGLVQPGGSLRLSCAASGFTFSSYAMHWVRQAPGKGLEWV | 47 |
| | AVISYDGSNKYYADSVKGRFTISRDNSKNTLYLQMNSLRAEDTAVYYC | |
| | ARFSSGWYYFDYWGQGTLVTVSSGGGGSGGGGSGGGGSDIQMTQSPSF | |
| | LSASVGDRITITCRASHDISSYFAWYQQKPGKAPKPLIYAASTLQSGV | |
| | PSRFSGSGSGTEFTLTISSLQPEDFATYYCQQLGSYPLTFGGGTKLEI | |
| | K | |
| M40pr146 | QVQLLQSGGGLVQPGGSLRLSCAASGFTFSSYAMSWVRQAPGKGLEWV | 48 |
| | SAISGSGGSTYYTDSVKGRFTISRDNSKNTLYLQMNSLRAEDTAVYYC | |
| | AKSHDYGDYAGFDYWGQGTLVTVSSGGGGSGGGGSGGGGSHVILTQDP | |
| | AVSVALGQTVRITCQGDSLKSYYASWYQQKPGQAPVLVIYGKNNRPSG | |
| | IPDRFSGSSSGTTASLTITGAQAEDEADYYCHSRDSSGTHLRVFGGGT | |
| | KLTVL | |
| UA20 | QVQLQESGGGLVKPGGSLRLSCAASGFTFSNAWMNWVRQAPGKGLEWV | 49 |
| | GRIKSKTDEGTTDYAAPVKGRESISRDDSKNTLYLQMNSLKTEDTGVY | |
| | YCTATKGLGGSKLGQGTLVTVSSGGGGSGGGGSGGGGSQSVLTQPPSA | |
| | SGTPGQRVTISCSGSSSNIGNNTVNWSRQLPGTAPKLLIYSNDQRPSG | |
| | VPDRFSGSKSGTSASLAITGLQPEDEADYYCGTWDSSLSAYVFGTGTK | |
| | LTVL | |
| UA8 | QVQLVESGGGVVQPGRSLRLSCAASGFTFSSFGMHWVRRAPGKGLEWV | 50 |
| | AVISYDGSNQYYADSVKGRFTISRDNSKNTLYLQMNSLRAEDTAVYYC | |
| | GSRPGGGYASGSTVAYWGQGTPVTVSSGGGGSGGGGSGGGGSSSELTQ | |
| | DPAVSVALGQTVRITCQGDSLRSYYASWYQQKPGQAPLLVIYGQNIRP | |
| | SGIPDRESGSSSGNSASLTITGAQAEDEADYYCHSRDSSGKYVFGVGT | |
| | KVTVL | |
| 585II41 | QVQLVESGGGLVQPGGSLRLSCAASGFTFSSYAMGWVRQAPGKGLEWV | 51 |
| | SAISGSGGSTYYADSVKGRFTISRDNSKDTLYLQMNSLRAEDTAVYYC | |
| | ASRSLLDYWGQGTLVTVSSGGGGSGGGGSGGGGSNFMLTQDPAVSVAL | |
| | GQTVRITCQGDSLRSYYASWYQQKPGQAPLLVIYGKNNRPSGIPDRES | |
| | GSSSGNTASLTITGAQAEDEADYYCNSRDSSGNPVFGGGTKVTVL | |
| 585II41.1 | QVQLVESGGGLVQPGGSLRLSCAASGFTFSSYAMSWVRQAPGKGLEWV | 52 |
| | SAISGSGGSTYYADSVKGRFTISRDNSKDTLYLQMNSLRAEDTAVYYC | |
| | ASRSLLDYWGQGTLVTVSSGGGGSGGGGSGGGGSNFMLTQDPAVSVAL | |
| | GQTVRITCQGDSLRSYYASWYQQKPGQAPLLVIYGKNNRPSGIPDRES | |
| | GSSSGNTASLTITGAQAEDEADYYCNSRDSSGNPVFGGGTKVTVL | |
| 585II56 | QVQLQESGGGLVQLGGSLRLSCAASGFTFSSYAMSWVRQAPGKGLEWV | 53 |
| | SAISGSGGSTYYADSVKGRFTISRDNSKNTLYLQMSSLRAEDTAFYYC | |
| | ANSAYTGGWYDYWGHGTLVTVSSGGGGSGGGGSGGGGSSSELTQDPAV | |
| | SVALGQTVKITCQGDSLRTYYASWYQQRPGQAPVLVIYGENSRPSGIP | |
| | DRFSGSSSGNTASLTITGAQAEDEADYYCNSRDSSGNHLRVFGGGTKL | |
| | TVL | |
| 3076 | QVNLRESGGGLVQPGGFLRLSCAAFGFTFSGYWMSWVHPAPGKGLEWV | 54 |
| | ANIKQDGSEKFYVDSVKGRFTISRDNAKNSLFLQMNSLRAEDTAVYFC | |
| | ARGLLSDYWGQGTLVPVSSGGGGSGGGGSGGGGSNFMLTQPPSVSVAP | |
| | GKTASLTCGGYNIGTKSVHWYQQKPGQAPVVVVHDDSDRPSGIPERFS | |
| | GSNSGTTATLTIIRVEAGDEADYYCQAWDSISEEVVFGGGTKLTVL | |
| 3051 | QVQLQESGGGLVKPGGPLRLSCAASGFTFSSYGMYWVRQAPGKGLEWV | 55 |
| | STLSRSGSGTYYAESVKGRFTISRDNSKNTLYFQMNSLRAEDTAVYYC | |
| | ASIAVAGNYFEYWGQGTLVTVSSGGGGSGGGGSGGGGSSYVLTQDPAV | |
| | SVALGQTVRITCQGDSLRSYYASWYQERPGQAPLLVIYGKNNRPSGIP | |
| | DRFSGSNSGSTATLTISRVEAGDEGDYYCQVWDSINEQVVFGGGTKVT | |
| | VL | |
| M49R | QVQLQESGGGLVKPGESLRLSCAASGFTFSDHYMDWVRQAPGKGLEWV | 56 |
| | AYIRYDGSTKYYADSVKGRFTISRDNSKNTLYLQMNSLRPEDTAFYYC | |
| | ARLIAEAEGWFDPWGQGTLVTVSSGGGGSGGGGSGGGGSNFMLTQPPS | |
| | VSVAPGKTARITCGGNNIGSKSVYWYQQKPGQAPVLVVYDDSDRPSGI | |
| | PERFSGSNSGNTATLTISRVEAGDEADYYCQVWDSSSDHVVFGGGTKV | |
| | TVL | |
| RCI-14 | QVQLLQSAGGLVQPGGSLRLSCAASGFTFSTYAMNWVRQAPGKGLEWV | 57 |
| | SGISGSGGSTNYADSVKGRFTISRDSSKNTLFLQMNSLRAEDTAVYYC | |
| | AKDYGSGWYDYWGQGTLVTVSSGGGGSGGGGSGGGGSSSELTQDPAVS | |
| | VALGQTVRITCQGDSLRSYYASWYQERPGQAPLLVIYGRNERPSGIPD | |
| | RFSASSSGNTASLTITGAQAEDEADYYCQVWDSFNEQVVFGGGTKLTV | |
| | L | |
| II79_4 | QVQLVESGGGLVQPGGSLRLSCAASGFTFSSYAMSWVHQAPGKGLEWV | 58 |
| | SAISGSGGSTYYADSVKGRFTISRDNSKNTLYLQMNSLRAEDTAVYYC | |
| | AKTYYGFWSGYYDYLGQGTLVTVSSGGGGsGGGGSGGGGSSSELTQDP | |
| | AVSVGLGQTVTITCQGDSLRSYYANWYQQKPGQAPILVIYGENNRPSG | |
| | IPDRFSGSSSGNTASLTITGAQAEDEADYYCHSRDSSGTHLRVFGGGT | |
| | KLTVL | |
| II79_3 | QVQLLESGGGVVQPGTSLRLSCAASGFTFSNYAINWVRQAAGKGLEWV | 59 |
| | SGISGSGVSTSYADSVKGRFTVSRDNSKNTLYLQMNSLRVEDTALYYC | |
| | AKNGGGPEYLQHWGQGTLVTVSSGGGGSGGGGSGGGGSQSVLTQPPSA | |
| | SGTPGQRVTISCSGSSSNIGNNTVNWSRQLPGTAPKLLIYSNDQRPSG | |
| | VPDRESGSKSGTSASLAITGLQPEDEADYYCGTWDSSLSAYVFGTGTK | |
| | LTVL | |
| T5II-4B.1 | QVQLQESGGTLVQPGGSLRLSCAASGFTFSSYAMSWVRQAPGRGLEWV | 60 |
| | STISGSGGSTYYADSVKGRFTISRDNSKNTLYLQMNSLRAEDTAVYYC | |
| | AKGAYSGSYWGQGTLVTVSSGGGGSGGGGSGGGGSSSELTQDPAVSVA | |
| | LGQTVRITCQGDSLRSYYASWYQQKPGQAPSLVIYGENSRPSGIPDRF | |
| | SGSSSGNTASLTITGAQAENEADYYCQAWDSSTAVVFGGGTKLTVL | |
| T5II-4B.2 | QVQLQESGGTLVQPGGSLRLSCAASGFTFSSYAMSWVRQAPGRGLEWV | 61 |
| | STISGSGGSTYYADSVKGRFTISRDNSKNTLYLQMNSLRAEDTAVYYC | |
| | AKGAYSGSHWGQGTLVTVSSGGGGSGGGGSGGGGSSSELTQDPAVSVA | |
| | LGQTVRITCQGDSLRSYYASWYQQKPGQAPSLVIYGENSRPSGIPDRF | |
| | SGSSSGNTASLTITGAQAENEADYYCQAWDSSTAVVFGGGTKLTVL | |
| RCI-11 | QVQLVESGAEVKKPGASVKVSCKASGYTFTSYGISWVRQAPGQGLEWM | 62 |
| | GWISAYNGNTNYAQKLQGRVTMTTDTSTSTAYMELRSLRSDDTAVYYC | |
| | ARPIYDSSGYDAFDIWGQGTMVTVSSGGGGSGGGGSGGGGSDIVMTQS | |
| | PSTLSASIGDRVTITCRASEGIYHWLAWYQQKPGKAPKLLIYKASSLA | |
| | SGAPSRFSGSGSGTDFTLTISSLQPDDFATYYCQQYHTISRTFGPGTK | |
| | VDIK | |
| RCI-20 | QVQLVESGGGLVKPGGSLRLSCAASGFTFSSYAMHWVRQAPGKGLEWV | 63 |
| | AVISYDGSNKYYADSVKGRFTISRDNSKNTLYLQMNSLRAEDTAVYFC | |
| | VRPSDSGWSFEHWGQGTLVPVSSGGGGSGGGGSGGGGSQSVLTQPPSA | |
| | SGTPGQRVTISCSGSSSNIGNNTVNWSRQLPGTAPKLLIYSNDQRPSG | |
| | VPDRFSGSKSGTSASLAITGLQPEDEADYYCGTWDSSLSAYVFGTGTK | |
| | LTVL | |
| CI-11A | QVQLQESGGGLVQPGGSLRLSCAASGFTFSSYAMSWVRQAPGKGLEWV | 64 |
| | AVISYDGSNKYYADSVKGRFTISRDNSKNTLYLQMNSLRAEDTAVYYC | |
| | VRGDRSYGAEYFQHWGQGTLVTVSSGGGGSGGGGSGGGGSSSELTQDP | |
| | AVSVASGQTVRITCQGDSLRSYYASWYQQKPGQAPLLVIYGKNIRPSG | |
| | IPDRESGSTSGNSASLTITGAQAEDEADYYCNSRDSSGNRNWVFGGGT | |
| | KLTVL | |
| CI-14A | QVQLQESGGGLVKPGGSLRLSCAASGFTSSSYAMHWVRQAPGKGLEYV | 65 |
| | SAIGGNGGTYYADSVKGRFTISRDNSKNTLYLQMNSLRAEDTAVYYCA | |
| | KEGEQWLEYRYYYGMDVWGQGTTVTVSSGGGGSGGGGSGGGGSSSELT | |
| | QDPAVSVALGQTVRITCQGDSLRSYYASWYQQKPGQAPSLVIYGENSR | |
| | PSGIPDRFSGSSSGNTASLTITGAQAENEADYYCQAWDSSTAVVFGGG | |
| | TKLTVL | |
| S95-2 | QVQLVESGGGVVQPGRSLRLSCTASGFTFSSYGMHWVRQAPGKGLEWV | 66 |
| | AVISYDGSNKYYADSVKGRFTISRDNSKNTLYLQMNSLRAEDTAVYYC | |
| | ARGGRYSSNWFSYYYYGMDVWGQGTTVTVSSGGGGSGGGGSGGGGSNF | |
| | MLTQPPSVSVAPGKTARITCGGNNIGSKSVYWYQQKPGQAPVLVVYDD | |
| | SDRPSGIPERFSGSNSGNTATLTISRVEAGDEADYYCQVWDSSSDHVV | |
| | FGGGTKVTVL | |

Using the amino acid sequences provided for the YS5, YS5F, YS5vlD, SB1HGNY, YS12, 3G7RY (aka 3G8), YS6, YS1, YS3, YS4, YS8, YS7, YS9, YS10, YS11, 3G7HY, 3G7NY, 3G7, SB32, 2C8, and UA8kappa antibodies, numerous antibody forms can be prepared, e.g., as described below. Such forms include, but are not limited to a substantially intact (e.g., full length) immunoglobulin (e.g., an IgA, IgE, IgG, and the like), an antibody fragment (e.g., Fv, Fab, (Fab′)₂, (Fab′)₃, IgGΔCH2, a minibody, and the like), a single chain antibody (e.g., scFv), a diabody, a unibody, an affibody, and the like.

It will be recognized, that where the antibodies are single chain antibodies, the VH and VL domains comprising such antibody can be joined directly together or by a peptide linker. Illustrative peptide linkers include, but are not limited to GGGGS GGGGS GGGGS (SEQ ID NO:67), GGGGS GGGGS (SEQ TD NO:68), GGGGS (SEQ TD NO:69), GS GGGGS GGGGS GGS GGGGS (SEQ TD NO:70), SGGGGS (SEQ TD NO:71), GGGS (SEQ TD NO:72), VPGV (SEQ TD NO:73), VPGVG (SEQ ID NO:74), GVPGVG (SEQ TD NO:75), GVG VP GVG (SEQ ID NO:76), VP GVG VP GVG (SEQ ID NO:77), GGSSRSS (SEQ ID NO:78), and GGSSRSSSSGGGGSGGGG (SEQ ID NO:79), and the like.

As indicated above, in various embodiments, the antibody binds (e.g., specifically binds CD46 (e.g., domains 1 and/or 2). Typically antibodies contemplated herein will specifically bind prostate cancer cells including, but not limited to cells of a cell line selected from the group consisting of DU145 cells, PC3 cells, and LnCaP cells. In certain embodiments the antibody binds to a prostate tumor cell with an affinity greater than (K_{D} less than) about 5 nM when measured on live prostate tumor cells by FACS. In certain embodiments the affinity is greater than (K_{D} less than) about 1 nM, or at about 100 pM, or about 50 pM, or about 10 pM, or about 1 pM.

Using the sequence information provided herein antibodies comprising one or more of the CDRs comprising, e.g., YS5, YS5F, YS5vlD, SB1HGNY, YS12, 3G7RY (aka 3G8), YS6, YS1, YS3, YS4, YS8, YS7, YS9, YS10, YS11, 3G7HY, 3G7NY, 3G7, SB2, 2C8, and UA8kappa, or antibodies comprising the VH and/or VL domain(s) of these antibodies can readily be prepared using standard methods (e.g. chemical synthesis methods and/or recombinant expression methods) well known to those of skill in the art, e.g., as described below.

In addition, other “related” prostate cancer specific antibodies can be identified by screening for antibodies that bind to the same epitope (e.g. that compete with one or more of YS5, YS5F, YS5vlD, SB1HGNY, YS12, 3G7RY (aka 3G8), YS6, YS1, YS3, YS4, YS8, YS7, YS9, YS10, YS11, 3G7HY, 3G7NY, 3G7, SB2, 2C8, and/or UA8kappa antibodies for binding to CD446 and/or to a cell expressing or overexpressing CD46, e.g., a prostate cancer cell) and/or by modification of the YS5, YS5F, YS5vlD, SB1HGNY, YS12, 3G7RY (aka 3G8), YS6, YS1, YS3, YS4, YS8, YS7, YS9, YS10, YS11, 3G7HY, 3G7NY, 3G7, SB2, 2C8, and/or UA8kappa antibodies identified herein to produce libraries of modified antibody and then rescreening antibodies in the library for improved binding to and/or internalization into cells expressing or overexpressing CD46, e.g., prostate cancer cells.

In some embodiments, that antibody is a recombinant antibody (or antigen binding fragment thereof) that specifically binds CD46. In some embodiments, antibody or antigen binding fragment or variant thereof is a monoclonal antibody. In some embodiments, antibody or antigen binding fragment or variant thereof is a human antibody, a murine antibody, a humanized antibody, or a chimeric antibody. In some embodiments, the antibody comprises or consists of a function fragment of a full length antibody (e.g., an antigen binding fragment of a full length antibody) such as a monovalent Fab, a bivalent Fab′2, a single-chain variable fragment (scFv), or functional fragment or variant thereof. In some embodiments, the recombinant antibody (or antigen binding fragment thereof) comprises an immunoglobulin variable heavy chain domain (VH). In some embodiments, the recombinant antibody (or antigen binding fragment thereof) comprises an immunoglobulin variable light chain domain (VL). In some embodiments, the recombinant antibody (or antigen binding fragment thereof) comprises a VH and a VL.

In some embodiments, the antibody (or antigen binding fragment thereof) comprises an Fc region. In some embodiments, the antibody (or antigen binding fragment thereof) is a full length antibody. In some embodiments, the antibody (or antigen binding fragment thereof) comprises a first light chain that comprises a light chain variable region and a light chain constant region; a first heavy chain that comprises a heavy chain variable region and a heavy chain constant region; a second light chain that comprises a light chain variable region and a light chain constant region; and a second heavy chain that comprises a heavy chain variable region and a heavy chain constant region. In some embodiments, the first and second light chains have at least 95%, 96%, 97%, 98%, 99%, or 100% sequence identity. In some embodiments, the first and second light chains bind the same epitope. In some embodiments, the first and second heavy chains have at least 95%, 96%, 97%, 98%, 99%, or 100% sequence identity. In some embodiments, the first and second heavy chains bind the same epitope.

In some embodiments, the antibody (or antigen binding fragment thereof) is derived from non-human (e.g. rabbit or mouse) antibodies. In some instances, the humanized form of the non-human antibody contains a minimal non-human sequence to maintain original antigenic specificity. In some cases, the humanized antibodies are human immunoglobulins (acceptor antibody), wherein the CDRs of the acceptor antibody are replaced by residues of the CDRs of a non-human immunoglobulin (donor antibody), such as rat, rabbit, or mouse donor having the desired specificity, affinity, avidity, binding kinetics, and/or capacity. In some instances, one or more framework region (FR) residues of the human immunoglobulin are replaced by corresponding non-human residues of the donor antibody.

In some embodiments, the CD46 binding antibody comprises an immunoglobulin variable heavy chain domain (VH) that comprises at least one, two, or three complementarity determining regions (CDRs) disclosed in Table 1, 2, or 3 or a sequence substantially identical thereto (e.g., a sequence that has at least 90%, 95%, 96%, 97%, 98%, or 99% sequence identity).

In some embodiments, the CD46 binding antibody comprises an immunoglobulin variable light chain domain (VL) that comprises at least one, two, or three complementarity determining regions (CDRs) disclosed in Table 1, 2 or 4 a sequence substantially identical thereto (e.g., a sequence that has at least 90%, 95%, 96%, 97%, 98%, or 99% sequence identity).

In some embodiments, the CD46 binding antibody comprises a VH that comprises at least one, two, or three complementarity determining regions (CDRs) disclosed in Table 1, 2, or 3 or a sequence substantially identical thereto (e.g., a sequence that has at least 90%, 95%, 96%, 97%, 98%, or 99% sequence identity); and a VL that comprises at least one, two, or three complementarity determining regions (CDRs) disclosed in Table 1, 2, or 4 or a sequence substantially identical thereto (e.g., a sequence that has at least 90%, 95%, 96%, 97%, 98%, or 99% sequence identity). For example, the CD46 binding antibody can comprise a VH that comprises at least one, two, or three complementarity determining regions (CDRs) disclosed in Table 3 or a sequence substantially identical thereto (e.g., a sequence that has at least 90%, 95%, 96%, 97%, 98%, or 99% sequence identity); and a VL that comprises at least one, two, or three complementarity determining regions (CDRs) disclosed in Table 4 or a sequence substantially identical thereto (e.g., a sequence that has at least 90%, 95%, 96%, 97%, 98%, or 99% sequence identity).

In some embodiments, the CD46 binding antibody comprises a VH that comprises a CDR1 of SEQ ID NO: 80, a CDR2 of SEQ ID NO: 81, and a CDR3 of SEQ ID NO: 82.

In some embodiments, the CD46 binding antibody comprises a VL that comprises a CDR1 of SEQ ID NO: 83, a CDR2 of SEQ ID NO: 84, and a CDR3 of SEQ ID NO: 85.

In some embodiments, the CD46 binding antibody comprises a VH that comprises a CDR1 of SEQ ID NO: 80, a CDR2 of SEQ ID NO: 81, and a CDR3 of SEQ ID NO: 82; and a VL that comprises a CDR1 of SEQ ID NO: 83, a CDR2 of SEQ ID NO: 84, and a CDR3 of SEQ ID NO: 85.

TABLE 3
VH CDR amino acid sequences of anti-CD46 antibodies as defined by
Kabat et al.

| | SEQ ID | | SEQ ID | | SEQ ID | |
|---|---|---|---|---|---|---|
| Antibody | NO | CDR1 | NO | CDR2 | NO | CDR3 |
| YS5FL | 80 | GLTVNNYA | 81 | ISYDGNNK | 82 | AKGGGYFDL |

Note
“YS5FL” and “YS5” refer to the same antibody, which differs at some positions from “YS5F”.

TABLE 4
VL CDR amino acid sequences of anti-CD46 antibodies as defined by
Kabat et al.

| | SEQ ID | | SEQ ID | | SEQ ID | |
|---|---|---|---|---|---|---|
| Antibody | NO | CDR1 | NO | CDR2 | NO | CDR3 |
| YS5FL | 83 | SSNIGAGYD | 84 | GNN | 85 | SSYTSGTWL |

YS5FL has been found to bind specifically to the surface of LnCap-C4-2B, LnCap-C4, DU145, PC3-luc, and Hs27 prostate cancer cells, but not to non-tumor BPH1 cells. Likewise, YS5FL binds specifically to the surface of RPMI8226, MM.1S, MM.1R, and INA6 multiple myeloma cells.

In some embodiments, a CDR described herein comprises one, two, or three amino acid modifications. In some embodiments, said modification is a substitution, addition, or deletion. In some embodiments, a CDR described herein comprises one, two, or three conservative amino acid substitutions. In some embodiments, the one, two, or three amino acid modifications does not substantially modify binding to human CD46. In some embodiments, the one, two, or three amino acid modifications modifies binding to human CD46. In some embodiments, a VH-CDR3 and/or VL CDR3 comprises an amino acid substitution that modifies binding to human CD46, immunogenicity, or some other feature. In some embodiments, the amino acid substitution is an alanine (A).

In some embodiments, the CD46 binding antibody comprises a VH that comprises an amino acid sequence disclosed in Table 5 or a sequence substantially identical thereto (e.g., a sequence that has at least 90%, 95%, 96%, 97%, 98%, or 99% sequence identity).

In some embodiments, the CD46 binding antibody comprises a VL that comprises an amino acid sequence disclosed in Table 6 or a sequence substantially identical thereto (e.g., a sequence that has at least 90%, 95%, 96%, 97%, 98%, or 99% sequence identity).

In some embodiments, the CD46 binding antibody comprises a VH that comprises an amino acid sequence disclosed in Table 5 or a sequence substantially identical thereto (e.g., a sequence that has at least 90%, 95%, 96%, 97%, 98%, or 99% sequence identity); and a VL that comprises an amino acid sequence disclosed in Table 6 or a sequence substantially identical thereto (e.g., a sequence that has at least 90%, 95%, 96%, 97%, 98%, or 99% sequence identity).

In some embodiments, the CD46 binding antibody comprises a VH that comprises an amino acid sequence of SEQ ID NO: 86, or a sequence substantially identical thereto (e.g., a sequence that has at least 90%, 95%, 96%, 97%, 98%, or 99% sequence identity).

In some embodiments, the CD46 binding antibody comprises a VL that comprises an amino acid sequence of SEQ ID NO: 87, or a sequence substantially identical thereto (e.g., a sequence that has at least 90%, 95%, 96%, 97%, 98%, or 99% sequence identity).

In some embodiments, the CD46 binding antibody comprises a VH that comprises an amino acid sequence of SEQ ID NO: 86, or a sequence substantially identical thereto (e.g., a sequence that has at least 90%, 95%, 96%, 97%, 98%, or 99% sequence identity); and a VL that comprises an amino acid sequence of SEQ ID NO: 87, or a sequence substantially identical thereto (e.g., a sequence that has at least 90%, 95%, 96%, 97%, 98%, or 99% sequence identity).

TABLE 5
Amino acid sequence of the anti-CD46 variable heavy chain binding
domains.

| | SEQ ID | |
|---|---|---|
| Name | NO | Amino Acid Sequence |
| YS5FL | 86 | QVQLVQSGGGVVQPGRSLRLACAASGLTVNNYAMHW |
| | | VRQAPGKGLEWVAVISYDGNNKYYADSVKGRFTISRD |
| | | NSKNTLYLQMNSLRAEDTAVYYCAKGGGYFDLWGRGTLVTVSS |

TABLE 6
Amino acid sequence of the anti-CD46 variable light chain
binding domains.

| | SEQ ID | |
|---|---|---|
| Name | NO | Amino Acid Sequence |
| YS5FL | 87 | QSVLTQPPSVSGAPGQRVTISCTGSSSNIGAGYDVHWYQQLPGT |
| | | APKLLIYGNNNRPSGVPDRESGSKSGTSASLAITGLQAEDEADY |
| | | YCSSYTSGTWLFGGGTKLTVL |

In some embodiments, the CD46 binding antibody comprises a heavy chain that comprises an amino acid sequence disclosed in Table 7 or a sequence substantially identical thereto (e.g., a sequence that has at least 90%, 95%, 96%, 97%, 98%, or 99% sequence identity).

In some embodiments, the CD46 binding antibody comprises a light chain that comprises an amino acid sequence disclosed in Table 8 or a sequence substantially identical thereto (e.g., a sequence that has at least 90%, 95%, 96%, 97%, 98%, or 99% sequence identity).

In some embodiments, the CD46 binding antibody comprises a heavy chain that comprises an amino acid sequence disclosed in Table 7 or a sequence substantially identical thereto (e.g., a sequence that has at least 90%, 95%, 96%, 97%, 98%, or 99% sequence identity); and a light chain that comprises an amino acid sequence disclosed in Table 8 or a sequence substantially identical thereto (e.g., a sequence that has at least 90%, 95%, 96%, 97%, 98%, or 99% sequence identity).

In some embodiments, CD46 binding antibody comprises a heavy chain that comprises an amino acid sequence of SEQ ID NO: 88, or a sequence substantially identical thereto (e.g., a sequence that has at least 90%, 95%, 96%, 97%, 98%, or 99% sequence identity).

In some embodiments, the CD46 binding antibody comprises a light chain that comprises an amino acid sequence of SEQ ID NO: 89, or a sequence substantially identical thereto (e.g., a sequence that has at least 90%, 95%, 96%, 97%, 98%, or 99% sequence identity).

In some embodiments, the CD46 binding antibody comprises a heavy chain that comprises an amino acid sequence of SEQ ID NO: 88, or a sequence substantially identical thereto (e.g., a sequence that has at least 90%, 95%, 96%, 97%, 98%, or 99% sequence identity); and a light chain that comprises an amino acid sequence of SEQ ID NO: 89, or a sequence substantially identical thereto (e.g., a sequence that has at least 90%, 95%, 96%, 97%, 98%, or 99% sequence identity).

TABLE 7
Amino acid sequence of the anti-CD46 heavy chain.

| | SEQ ID | |
|---|---|---|
| Name | NO | Amino Acid Sequence |
| YS5FL | 88 | QVQLVQSGGGVVQPGRSLRLACAASGLIVNNYAMHWVRQAPGK |
| | | GLEWVAVISYDGNNKYYADSVKGRFTISRDNSKNTLYLQMNSL |
| | | RAEDTAVYYCAKGGGYFDLWGRGTLVTVSSASTKGPSVFPLAP |
| | | SSKSTSGGTAALGCLVKDYFPEPVTVSWNSGALTSGVHTFPAV |
| | | LQSSGLYSLSSVVTVPSSSLGTQTYICNVNHKPSNTKVDKKVE |
| | | PKSCDKTHTCPPCPAPELLGGPSVFLFPPKPKDTLMISRTPEV |
| | | TCVVVDVSHEDPEVKFNWYVDGVEVHNAKTKPREEQYNSTYRV |
| | | VSVLTVLHQDWLNGKEYKCKVSNKALPAPIEKTISKAKGQPRE |
| | | PQVYTLPPSREEMTKNQVSLTCLVKGFYPSDIAVEWESNGQPE |
| | | NNYKTTPPVLDSDGSFFLYSKLTVDKSRWQQGNVFSCSVMHEA |
| | | LHNHYTQKSLSLSPGK |

TABLE 8
Amino acid sequence of the anti-CD46 light chain.

| | SEQ ID | |
|---|---|---|
| Name | NO | Amino Acid Sequence |
| YS5FL | 89 | QSVLTQPPSVSGAPGQRVTISCTGSSSNIGAGYDVHWYQQLPGT |
| | | APKLLIYGNNNRPSGVPDRESGSKSGTSASLAITGLQAEDEADY |
| | | YCSSYTSGTWLFGGGTKLTVLGQPKAAPSVTLFPPSSEELQANK |
| | | ATLVCLISDFYPGAVTVAWKADSSPVKAGVETTTPSKQSNNKYA |
| | | ASSYLSLTPEQWKSHRSYSCQVTHEGSTVEKTVAPTECS |

In some embodiments, the anti-CD46 antibody disclosed herein comprises an immunoglobulin constant region (e.g., an Fc region). Exemplary Fc regions can be chosen from the heavy chain constant regions of IgG1, IgG2, IgG3 or IgG4; more particularly, the heavy chain constant region of human IgG1 or IgG4. In some embodiments, the immunoglobulin constant region (e.g., the Fc region) is altered, e.g., mutated, to increase or decrease one or more of: Fc receptor binding, antibody glycosylation, the number of cysteine residues, effector cell function, or complement function.

In some embodiments, disclosed herein are immunoconjugates that comprise an anti-CD46 antibodies attached to an effector agent (or prodrug thereof). In some embodiments, the effector agent is a drug (or prodrug thereof), small molecule, protein, peptide, antibody, ligand, receptor, cytotoxic agent, cytostatic agent, liposome, nanoparticle, radionuclide, cytokine, chemokine, a toxin, a detectable label, a viral particle, or a chelate.

In some embodiments, the effector agent is a drug (or prodrug thereof). In some embodiments, the effector agent is an anti-cancer agent (or prodrug thereof). In some embodiments, the effector agent is a chemotherapeutic agent (or prodrug thereof). In some embodiments, the effector agent is a microtubule inhibitor (or prodrug thereof), a DNA-damaging agent (or prodrug thereof), or a polymerase inhibitor (or prodrug thereof).

In some embodiments, the effector agent is a microtubule inhibitor (or prodrug thereof). In some embodiments, the microtubule inhibitor is an auristatin (or a derivative thereof), dolastatin-10 (or a derivative thereof), or maytansine (or a derivative thereof). In some embodiments, the microtubule inhibitor is monomethylauristatin F (MMAF), auristatin E (AE), monomethylauristatin E (MMAE), valine-citrulline MMAE (vcMMAE), or valine-citrulline MMAF (vcMMAF). In some embodiments, the microtubule inhibitor is monomethylauristatin E (MMAE).

In some embodiments, the effector agent comprises or consists of a compound of Formula A:

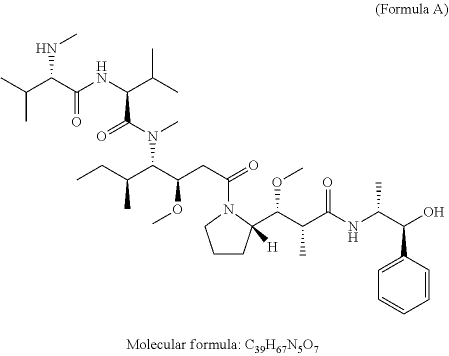 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@@H](NC)C(C)C)C(C)C]

In certain embodiments, the effector comprises a detectable label. Suitable detectable labels include, but are not limited to radio-opaque labels, nanoparticles, PET labels, MRI labels, radioactive labels, and the like. Among the radionuclides and useful in various embodiments, gamma-emitters, positron-emitters, x-ray emitters and fluorescence-emitters are suitable for localization, diagnosis and/or staging, and/or therapy, while beta and alpha-emitters and electron and neutron-capturing agents, such as boron and uranium, also can be used for therapy.

In one aspect, provided herein are immunoconjugates comprising an anti-CD46 antibody and an effector agent. In some embodiments, the methods described herein utilize these immunoconjugates.

In some embodiments, the immunoconjugate comprises an anti-CD46 antibody (or antigen binding fragment thereof) described herein. In some embodiments, the immunoconjugate comprises a YS5FL antibody (or antigen binding fragment thereof).

In some embodiments, the effector agent is conjugated to the anti-CD46 antibody. In some embodiments, the effector agent is attached to the anti-CD46 antibody via a liker. In some embodiments, the linker is a peptide linker, a small molecule linker, or a linker that comprises a peptide and a small molecule. Exemplary peptide linkers include, but are not limited to, peptide linkers comprising glycine, serine, or glycine and serine.

In some embodiments, the linker is cleavable. In some embodiments, the linker is cleaved only upon internalization into a cell. In some embodiments, the cleavable linker is only cleavable upon internalization into a cancer cell. In some embodiments, the cleavable portion of a linker is a peptide (e.g., a dipeptide, e.g., ValCit). In some embodiments, the cleavable linker is cleavable by cathepsin. In some embodiments, the linker comprises maleimide. In some embodiments, the linker comprises caproic acid. In some embodiments, the linker comprises maleimide and caproic acid. In some embodiments, the linker comprises maleimide, caproic acid, and a cleavable dipeptide.

In some embodiments, the linker comprises or consists of is a maleimidocaproyl-valinecitrulline-para-amino benzyloxycarbonyl (mc-vc-PAB).

In some embodiments, the linker comprises or consists of a compound of Formula B:

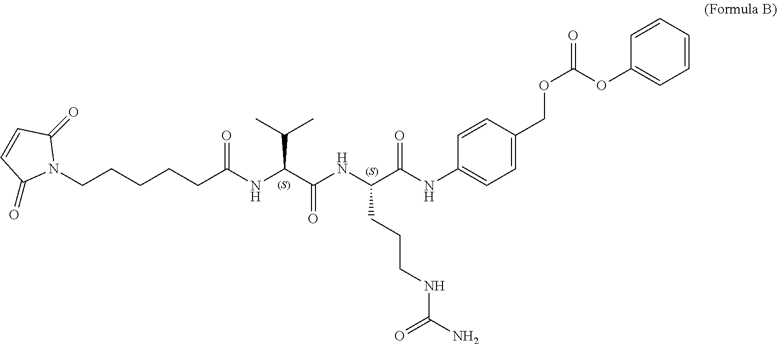

In some embodiments, an effector agent is attached to a light chain of the anti-CD46 antibody. In some embodiments, an effector agent is attached to a light chain constant region of the anti-CD46 antibody. In some embodiments, an effector agent is attached to a heavy chain of the anti-CD46 antibody. In some embodiments, an effector agent is attached to a heavy chain constant region of the anti-CD46 antibody.

In some embodiments, an effector moiety is attached to a cysteine residue of the anti-CD46 antibody. In some embodiments, an anti-CD46 antibody is partially reduced prior to conjugation to an effector moiety such that 1-4 interchain disulfide bonds are reduced while intrachain disulfide bonds are not reduced. Partial reduction exposes pairs of cysteine residues, rendering them accessible to conjugation to adducts such as mc-vc-PAB-MMAE. In some embodiments, the following interchain cysteine pairs of YS5FL are exposed: C219 of the first heavy chain and C214 of the first light chain; C219 of the second heavy chain and C214 of the second light chain; C225 of the first heavy chain and C225 of the second light chain; and C228 of the first heavy chain and C228 of the second light chain. In some embodiments, an effector such as mc-vc-PAB-MMAE is conjugated to 0, 1, 2, 3, or 4 pairs of cysteine residues on YS5FL.

In some embodiments, the ratio of effector agents to anti-CD46 antibodies is 1:1, 2:1, 3:1, 4:1, 5:1, 6:1, 7:1 or 8:1. In some embodiments, the ratio of effector agents to anti-CD46 antibodies is 2:1, 4:1, 6:1, or 8:1. In some embodiments, the ratio of effector agents to anti-CD46 antibodies is about 4:1. In some embodiments, the average ratio of effector agents to anti-CD46 antibodies is about 3.7:1. In some embodiments, if the immunoconjugate comprises 2 or more effector agents, each effector agent is the same. In some embodiments, if the immunoconjugate comprises 2 or more effector agents, at least two effector agents are different. In some embodiments, the ratio of effector agents to anti-CD46 antibodies is about 4:1 and each effector agent is the same.

An exemplary immunoconjugate provided herein comprises an anti-CD46 YS5FL antibody linked to a monomethyl auristatin E (MMAE) effector agent via a maleimidocaproyl-valine-citrullinepara-amino benzyloxycarbonyl (mc-vc-PAB). In some embodiments, the ratio of MMAE to YSFL antibody is about 4:1.

In some embodiments, the immunoconjugate comprises the antibody conjugate below in Formula C, wherein the comprises heavy chain of SEQ ID NO: 9; and a light chain of SEQ ID NO: 10. This immunoconjugate is also referred to herein as FOR46 and comprises YS5FL antibody attached to MMAE through a mc-vc-PAB linker.

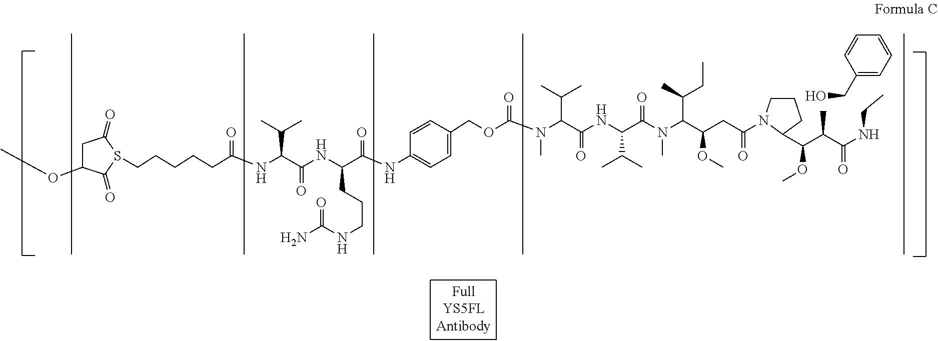

In some embodiments, an anti-CD46 immunoconjugate described herein is manufactured by a process comprising reduction or partial reduction of disulfide bonds of an immunoglobulin. In some embodiments, an anti-CD46 immunoconjugate described herein is manufactured by a process comprising reduction or partial reduction of interchain disulfide bonds of an immunoglobulin. In some embodiments, the reducing agent is dithiothreitol (DTT) or tris(2-carboxyethyl)phosphine (TCEP). In some embodiments, an effector-linker complex comprising a maleimide reactive group is conjugated to pairs of reduced cysteines of an immunoglobulin. In some embodiments, the effector-linker complex is mc-vc-PAB-MMAE.

In some embodiments, an effector-linker complex is conjugated at C219, C225, or C228 of a YS5FL heavy chain (SEQ ID NO: 9) or C214 of a YS5FL light chain (SEQ ID NO: 10), or any combination thereof. In some embodiments, the effector-linker complexes are conjugated to C219 of a YS5FL heavy chain and C214 of a YS5FL light chain. In some embodiments, an anti-CD46 immunoconjugate comprises two YS5FL heavy chains and two YS5FL light chains and effector-linker complexes are conjugated to C219 of a YS5FL first heavy chain, C214 of a first YS5FL light chain, C219 of a YS5FL second heavy chain, and C214 of a second YS5FL light chain. In some embodiments, an anti-CD46 immunoconjugate comprises two YS5FL heavy chains and an effector-linker complex is conjugated to C225 of a first YS5FL heavy chain and C225 of a second YS5FL heavy chain. In some embodiments, an anti-CD46 immunoconjugate comprises two YS5FL heavy chains and an effector-linker complex is conjugated to C228 of a first YS5FL heavy chain and C228 of a second YS5FL heavy chain. In some embodiments, an immunoconjugate comprises two, four, six, or eight effectors and the effectors are conjugated to any one, two, three, or four, respectively, of the following pairs of cysteines: C219 of HC1 and C214 of LC1; C219 of HC2 and C214 of LC2; C225 of HC1 and C225 of HC2; and C228 of HC1 and C228 of HC2.

In some embodiments, purified YS5FL mAb (e.g., 10 mg/ml) can adjusted to a pH of 6.8 with sodium phosphate buffer and then treated with TCEP (e.g., TCEP/mAb ratio of 2.1) for two hours at 22° C. Reduced mAb can be reacted with mc-vc-PAB-MMAE (e.g., drug/mAb ratio of 6) in 9% dimethylacetamide for 15 minutes. In some embodiments, the mAb can reduced a second time for one hour, conjugated a second time for 60 min, and the reaction can quenched, e.g., by lowering the pH to 5.0 with 1M acetic acid, yielding a immunoconjugate, for example with a drug to antibody ratio of about 3.7, as determined by hydrophobic interaction chromatography.

In some embodiments, an anti-CD46 antibody or immunoconjugate described herein binds to CD46 expressed on the surface of a target cell (e.g., a cancer cell) and is internalized by the cell. In some embodiments, the antibody or immunoconjugate is internalized into the target cell via macropinocytosis. In some embodiments, the antibody or immunoconjugate is targeted to a lysosome of the cell upon internalization. In some embodiments, the antibody or immunoconjugate induces internalization into the cell without crosslinking.

In some embodiments, an anti-CD46 antibody or immunoconjugate described herein mediates killing of a target cell (e.g., cancer cell) upon internalization. In some embodiments, the anti-CD46 antibody or immunoconjugate induces apoptosis of the target cell (e.g., cancer cell) upon internalization. In some embodiments, the anti-CD46 antibody or immunoconjugate inhibits cell division of the target cell (e.g., cancer cell) upon internalization. In some embodiments, the anti-CD46 antibody or immunoconjugate selectively inhibits cell division of cancer cells upon internalization and does not inhibit cell division of non-cancer cells upon internalization.

In some embodiments, antibodies (and antigen binding fragment thereof) are produced using any method known in the art to be useful for the synthesis of antibodies, in particular, by chemical synthesis or by recombinant expression techniques.

In some embodiments, an antibody (or antigen binding fragment thereof) is expressed recombinantly. In some embodiment, the nucleic acid encoding the antibody (or antigen binding fragment thereof) is assembled from chemically synthesized oligonucleotides. In some embodiments, a nucleic acid molecule encoding an antibody is generated from a suitable source (e.g., an antibody cDNA library, or cDNA library generated from any tissue or cells expressing the immunoglobulin) by PCR amplification using synthetic primers hybridizable to the 3′ and 5′ ends of the sequence or by cloning using an oligonucleotide probe specific for the particular gene sequence.

In some embodiments, an antibody (or antigen binding fragment thereof) is made by immunizing an animal, such as a mouse, to generate polyclonal or monoclonal antibodies.

In some embodiments, an expression vector comprising the nucleotide sequence of an antibody or the nucleotide sequence of an antibody is transferred to a host cell by conventional techniques (e.g., electroporation, liposomal transfection, and calcium phosphate precipitation), and the transfected cells are then cultured by conventional techniques to produce the antibody. In some embodiments, the expression of the antibody is regulated by a constitutive, an inducible or a tissue, specific promoter.

A variety of host-expression vector systems can be utilized to express an antibody (or antigen binding fragment thereof) described herein. These include, but are not limited to, microorganisms such as bacteria (e.g., E. coli and B. subtilis) transformed with recombinant bacteriophage DNA, plasmid DNA or cosmid DNA expression vectors containing an antibody or its binding fragment coding sequences; yeast (e.g., Saccharomyces Pichia) transformed with recombinant yeast expression vectors containing an antibody or its binding fragment coding sequences; insect cell systems infected with recombinant virus expression vectors (e.g., baculovirus) containing an antibody or its binding fragment coding sequences; plant cell systems infected with recombinant virus expression vectors (e.g., cauliflower mosaic virus (CaMV) and tobacco mosaic virus (TMV)) or transformed with recombinant plasmid expression vectors (e.g., Ti plasmid) containing an antibody or its binding fragment coding sequences; or mammalian cell systems (e.g., COS, CHO, BH, 293, 293T, 3T3 cells) harboring recombinant expression constructs containing promoters derived from the genome of mammalian cells (e.g., metallothionein promoter) or from mammalian viruses (e.g. the adenovirus late promoter; the vaccinia virus 7.5K promoter).

For long-term, high-yield production of recombinant proteins, stable expression may be preferred. In some embodiments, cell lines that stably express an antibody are made. Following the introduction of the foreign DNA, engineered cells are then allowed to grow for 1-2 days in an enriched media, and then are switched to a selective media. A selectable marker in the recombinant plasmid may be used to confer resistance to the selection.

In some embodiments, any method known in the art for purification of an antibody can be used, for example, by chromatography (e.g., ion exchange, affinity, particularly by affinity for the specific antigen after Protein A, and sizing column chromatography), centrifugation, differential solubility, or by any other standard technique for the purification of proteins.

IMiDs used in the methods and compositions described herein can be selected, for example, from those known in the art.

In one embodiment, the IMiD includes thalidomide or analogs thereof. Thalidomide is registered under the trade name Thalidomid™ (Celgene Corp). Thalidomide and analogs thereof, and methods of making the same, are known, for example, as described in U.S. Pat. Nos. 6,045,501; 7,230,012; 7,435,745.

In another embodiment, the IMiD includes pomalidomide or analogs thereof. Pomalidomide is registered under the trade name Pomalyst™ (Celgene Corp). Pomalidomide and analogs thereof, and methods of making the same, are known, for example, as described in U.S. Pat. Nos. 5,635,517; 6,316,471; 6,476,052; 8,158,653; 8,198,262; 8,673,939; 8,735,428; and 8,828,427.

In another embodiment, the IMiD includes lenalidomide or analogs thereof. Lenalidomide is registered under the trade name Revlimid™ (Celgene Corp). Lenalidomide and analogs thereof, and methods of making the same are known, for example, as described in U.S. Pat. Nos. 5,635,517; 6,555,554; 7,119,106; 7,465,800; 7,855,217; 8,288,415; and 8,530,498.

In another embodiment, the IMiD is apremilast or analogs thereof. Apremilast is registered under the trade name Otezla™ (Celgene Corp). Apremilast and analogs thereof, and methods of making the same, are known to those skilled in the art, for example, those described in U.S. Pat. Nos. 6,020,358; 7,427,638; 7,893,101.

In another embodiment, the IMiD is iberdomide or analogs thereof. Iberdomide structure is provided in e.g., US 20200330445.

Stat3 inhibitors used in the methods and compositions described herein can be selected, for example, from those known in the art. Exemplary Stat3 inhibitors include but are not limited to N-(1′, 2-Dihydroxy-1,2′-binaphthalen-4′-yl)-4-methoxybenzenesulfonamide (C188-9), STAT3 Inhibitor V, 6-Nitrobenzo[b]thiophene 1,1-dioxide (Stattic), (1E,6E)-1,7-Bis(4-hydroxy-3-methoxyphenyl)-1,6-heptadiene-3,5-dione (curcumin), N-Hexyl-2-(1-naphthalenyl)-5-[[4-(phosphonooxy)phenyl]methyl]-4-oxazolecarboxamide (S3I-M2001), 8-hydroxy-3-methyl-3,4-dihydrotetraphene-1,7,12(2H)-trione (STA-21), 2-Hydroxy-4-[[2-[[(4-methylphenyl)sulfonyl]oxy]acetyl]amino]benzoic acid (S3I-201), Cepharanthine, Cucurbitacin I, Cucumis sativus L, Niclosamide, Cryptotanshinone, SD 1008, Stat3 Inhibitor III, WP1066, Nifuroxazide, Stat3 Inhibitor VI, S31-201, STA-21, Kahweol, STAT3 Inhibitor IX, Cpd188; STAT3 Inhibitor VI, S31-201; STAT3 Inhibitor VII Ethyl-1-(4-cyano-2,3,5,6-tetrafluorophenyl)-6,7,8-trifluoro-4-oxo-1,4-dihydroquinoline-3-carboxylate; STAT3 Inhibitor VIII, 5,15-DPP, STAT3 Inhibitor X, HJB; STAT3 Inhibitor XII, SPI; STAT3 Inhibitor XI, STX-0119; STAT3 Inhibitor XIV, LLL12; FLLL32; FLLL62; Napabucasin (BB1608); DSP-0337 (prodrug of napabucasin); OPB-51602; OPB-31121; OPB-111077; Pyrimethamine; WP1066 or derivatives or analogues or salts thereof.

In some embodiments, one or more selective glucocorticoid receptor agonist or modulator (SEGRAM) are also administered to the human. Selective glucocorticoid receptor agonists (SEGRAs) are historically and typically steroidal in structure while selective glucocorticoid receptor modulators (SEGRMs) are typically nonsteroidal. The latter class is able to modulate the activity of a GR agonist and/or may not classically bind the glucocorticoid receptor ligand-binding pocket. See, e.g., Sundahl et al, Pharmacology & Therapy, Volume 152, August 2015, Pages 28-41. The combined abbreviation of selective glucocorticoid receptor agonist or modulator is SEGRAM. Exemplary SEGRAs include, for example, dexamethasone, prednisone, and cortisol. Exemplary SEGRMs include, for example, mapracorat, fosdagrocorat (PF-04171327), and dagrocorat.

Pharmaceutically acceptable forms of iMiD, Stat3 inhibitor or SEGRAM compounds, such as free base, salts, polymorphs, solvates, solutions, isomers, amorphous, crystalline, co crystalline, solid solution, prodrugs, analogs, derivatives, and metabolites are contemplated for use in the methods described herein. The compound may be in the form of a pharmaceutically acceptable salt, such as an acid addition salt or a base salt, or a solvate thereof, including a hydrate thereof. Suitable acid addition salts are formed from acids which form non-toxic salts and examples are the hydrochloride, hydrobromide, hydroiodide, sulphate, bisulphate, nitrate, phosphate, hydrogen phosphate, acetate, maleate, fumarate, lactate, tartrate, citrate, gluconate, succinate, saccharate, benzoate, methanesulphonate, ethanesulphonate, benzenesulphonate, p-toluenesulphonate and pamoate salts.

In one aspect, provided herein are methods of treating certain cancers by administering to a human an IMiD or Stat3 inhibitor (and optionally a SEGRAM, or both an IMiD and a SEGRAM or both a Stat3 inhibitor and a SEGRAM, administered either sequentially or in combination) as well as an anti-CD46 antibody or immunoconjugate described herein.

In some embodiments, the cancer is multiple myeloma. In some embodiments, the cancer is relapsing multiple myeloma. In some embodiments, the cancer is remitting multiple myeloma. In some embodiments, the cancer is relapsing or remitting multiple myeloma.

In some embodiments, the cancer is prostate cancer. In some embodiments, the cancer is castration resistant prostate cancer. In some embodiments, the cancer is metastatic prostate cancer.

In some embodiments, the cancer is lymphoma (including but not limited to Hodgkin's lymphoma), acute myeloid leukemia (AML), or metastatic renal cell carcinoma (mRCC).

In some embodiments, an agent that induces expression of CD46, wherein the agent is an immunomodulatory imide drug (IMiD) and optionally a glucocorticoid receptor agonist or modulator (SEGRAM), is administered to a human having a cancer as described herein for a first period of time sufficient to result in increased expression of CD46 in cancer cells, followed by administration of an anti-CD46 antibody or immunoconjugate described herein for a second period of time after the first period of time. In some embodiments, an agent that induces expression of CD46, wherein the agent is a Stat3 inhibitor and optionally an immunomodulatory imide drug (IMiD) or a glucocorticoid receptor agonist or modulator (SEGRAM) or both, is administered to a human having a cancer as described herein for a first period of time sufficient to result in increased expression of CD46 in cancer cells, followed by administration of an anti-CD46 antibody or immunoconjugate described herein for a second period of time after the first period of time. In some embodiments, the agent can be co-administered with the anti-CD46 antibody or immunoconjugate in the second time period. In some embodiments, for example, the first period is (e.g., once or more a day for) 2-20 days, e.g., 5-15 days, e.g., 5-10 days, e.g., 3-10 days, e.g., 2, 3, 4, 5, 6, 7, 8, 9, 10 days. In some embodiments, the second period is (e.g., once or more a day for) 1-90 days, e.g., 1-60, 15-45, 5-15 days, e.g., 5-10 days, e.g., 3-10 days, e.g., 1, 2, 3, 4, 5, 6, 7, 8, 9, 10, 20, 30, 40, or 50 days. In some embodiments, an IMiD or Stat3 inhibitor is administered to a human having cancer as described herein in the absence of an anti-CD46 antibody for the first period. In some embodiments, the IMiD is selected from pomalidamide, lenolidamide, thalidomide, iberdomide, and apremilast. In some embodiments, a SEGRAM such as a glucocorticoid receptor agonist is also administered to a human having cancer as described herein in the absence of an anti-CD46 antibody for the first period. In some embodiments, the SEGRAM is selected from dexamethasone, prednisone, cortisol, mapracorat, fosdagrocorat (PF-04171327), and dagrocorat. In some embodiments, an IMiD and a SEGRAM are administered to a human having cancer as described herein in the absence of an anti-CD46 antibody for the first period. In some embodiments, in the second period the anti-CD46 antibody or immunoconjugate is administered with the immunomodulatory imide drug (IMiD) or a glucocorticoid receptor agonist or modulator (SEGRAM) or both or with a Stat3 inhibitor, or both a Stat3 inhibitor and a SEGRAM. In some embodiments, in the second period the amount (e.g., concentration or final amount) of IMiD or Stat3 inhibitor administered is the same or is less than the amount (e.g., concentration or final amount) administered in the first period of time. In some embodiments, in the second period the anti-CD46 antibody or immunoconjugate is administered alone or with the glucocorticoid receptor agonist or modulator (SEGRAM), but not the IMiD or Stat3 inhibitor. In some embodiments, in the second period the anti-CD46 antibody is administered with the IMiD. In some embodiments, in the second period the anti-CD46 antibody is administered a different IMiD than was administered in the first period (e.g., pomalidomide in the first period and lenalidomide in the second period, or vice versa). In some embodiments, in the second period the anti-CD46 antibody is administered with the Stat3 inhibitor.

In one example, pomalidomide is administered to the human for 5-10 days (e.g., 7 days) at 4 mg daily, followed by a combination in a second period of pomalidomide (4 mg daily) and dexamethasone 40 mg daily, or 20 mg daily if human is over 75 years old) and an anti-CD46 immunoconjugate comprising a cytotoxin (1.8-2.7 mg/kg daily). In another example, pomalidomide is administered to the human for 5-10 days (e.g., 7 days) at 4 mg daily, followed by a combination in a second period of lenalidomide (4 mg daily) and dexamethasone 40 mg daily, or 20 mg daily if human is over 75 years old) and an anti-CD46 immunoconjugate comprising a cytotoxin (1.8-2.7 mg/kg daily). In another example, pomalidomide or lenalidomide is administered to the human for 5-10 days (e.g., 7 days) at 4 mg daily, followed by in a second period an anti-CD46 immunoconjugate comprising a cytotoxin (1.8-2.7 mg/kg daily) without an IMiD, and optionally with dexamethasone.

Pharmaceutical compositions as described herein (e.g., anti-CD46 antibodies, IMiDs, etc.) can be prepared in accordance with methods well known and routinely practiced in the art. Pharmaceutically acceptable carriers are determined in part by the particular composition being administered, as well as by the particular method used to administer the composition. Accordingly, there is a wide variety of suitable formulations of pharmaceutical compositions described herein. Applicable methods for formulating the antibodies and determining appropriate dosing and scheduling can be found, for example, in Remington: The Science and Practice of Pharmacy, 21^{st} Ed., University of the Sciences in Philadelphia, Eds., Lippincott Williams & Wilkins (2005); and in Martindale: The Complete Drug Reference, Sweetman, 2005, London: Pharmaceutical Press., and in Martindale, Martindale: The Extra Pharmacopoeia, 31st Edition., 1996, Amer Pharmaceutical Assn, and Sustained and Controlled Release Drug Delivery Systems, J. R. Robinson, ed., Marcel Dekker, Inc., New York, 1978, each of which are hereby incorporated herein by reference. Pharmaceutical compositions are preferably manufactured under GMP conditions. Typically, a therapeutically effective dose or efficacious dose of the antibody or other compounds descried herein is employed in the pharmaceutical compositions. The antibodies can be formulated into pharmaceutically acceptable dosage forms by conventional methods known to those of skill in the art. Dosage regimens are adjusted to provide the desired response (e.g., a therapeutic response). In determining a therapeutically or prophylactically effective dose, a low dose can be administered and then incrementally increased until a desired response is achieved with minimal or no undesired side effects. For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier.

Actual dosage levels of the active ingredients in the pharmaceutical compositions can be varied so as to obtain an amount of the active ingredient which is effective to achieve the desired therapeutic response for a particular patient, composition, and mode of administration, without being toxic to the patient. The selected dosage level depends upon a variety of pharmacokinetic factors including the activity of the particular compositions of the present invention employed, or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors.

A therapeutically effective amount of the antibodies and compounds will vary depending upon the subject and disease condition being treated, the weight and age of the subject, the severity of the disease condition, the manner of administration and the like, which can readily be determined by one of ordinary skill in the art. The dosages for administration can range from, for example, about 1 ng to about 10,000 mg, about 5 ng to about 9,500 mg, about 10 ng to about 9,000 mg, about 20 ng to about 8,500 mg, about 30 ng to about 7,500 mg, about 40 ng to about 7,000 mg, about 50 ng to about 6,500 mg, about 100 ng to about 6,000 mg, about 200 ng to about 5,500 mg, about 300 ng to about 5,000 mg, about 400 ng to about 4,500 mg, about 500 ng to about 4,000 mg, about 1 μg to about 3,500 mg, about 5 μg to about 3,000 mg, about 10 μg to about 2,600 mg, about 20 μg to about 2,575 mg, about 30 μg to about 2,550 mg, about 40 μg to about 2,500 mg, about 50 μg to about 2,475 mg, about 100 μg to about 2,450 mg, about 200 μg to about 2,425 mg, about 300 μg to about 2,000, about 400 μg to about 1,175 mg, about 500 μg to about 1,150 mg, about 0.5 mg to about 1,125 mg about 1 mg to about 1,100 mg, about 1.25 mg to about 1,075 mg, about 1.5 mg to about 1,050 mg, about 2.0 mg to about 1,025 mg, about 2.5 mg to about 1,000 mg, about 3.0 mg to about 975 mg, about 3.5 mg to about 950 mg, about 4.0 mg to about 925 mg, about 4.5 mg to about 900 mg, about 5 mg to about 875 mg, about 10 mg to about 850 mg, about 20 mg to about 825 mg, about 30 mg to about 800 mg, about 40 mg to about 775 mg, about 50 mg to about 750 mg, about 100 mg to about 725 mg, about 200 mg to about 700 mg, about 300 mg to about 675 mg, about 400 mg to about 650 mg, about 500 mg, or about 525 mg to about 625 mg, e.g., 1 to 10 mg/kg, 1.8 to 2.7 mg/kg of an anti-CD46 antibody described herein and/or antigen binding portion thereof, and/or immunoconjugate thereof as described herein. Dosage regiments may be adjusted to provide the optimum therapeutic response. An effective amount is also one in which any toxic or detrimental effects (i.e., side effects) of an antibody or antigen binding portion thereof are minimized and/or outweighed by the beneficial effects. Exemplary dosages for IMiDs can be determined as known in the art. Exemplary IMiD dosages can be for example, 1-10 mg per dose per day. Exemplary SEGRAM (e.g., dexamethasone) dosages can be for example, 0.1-60 (e.g., 0.1-20 or 20 or 40 mg) mg per dose per day.

Compositions described herein can be formulated as a pharmaceutically acceptable salt, i.e., a biologically compatible salt of a disclosed compound, which salts are derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, tartrate, mesylate, acetate, maleate, oxalate, and the like. Pharmaceutically acceptable acid addition salts are those salts that retain the biological effectiveness of the free bases while formed by acid partners that are not biologically or otherwise undesirable, e.g., inorganic acids such as hydrochloric acid, hydrobromic acid, sulfuric acid, nitric acid, phosphoric acid, and the like, as well as organic acids such as acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid and the like. Pharmaceutically acceptable base addition salts include those derived from inorganic bases such as sodium, potassium, lithium, ammonium, calcium, magnesium, iron, zinc, copper, manganese, aluminum salts and the like. Exemplary salts are the ammonium, potassium, sodium, calcium, and magnesium salts. Salts derived from pharmaceutically acceptable organic non-toxic bases include, but are not limited to, salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins, and the like. Exemplary organic bases are isopropylamine, diethylamine, ethanolamine, trimethylamine, dicyclohexylamine, choline, and caffeine. See, for example, S. M. Berge, et al., “Pharmaceutical Salts,” J. Pharm. Sci., 1977; 66:1-19, which is incorporated herein by reference. For therapeutic use, salts of the compounds are those wherein the counter-ion is pharmaceutically acceptable. However, salts of acids and bases which are non-pharmaceutically acceptable may also find use, for example, in the preparation or purification of a pharmaceutically acceptable compound.

Pharmaceutical compositions as described herein can be administered by a variety of methods known in the art. The route and/or mode of administration vary depending upon the desired results. It is preferred that administration be intravenous, intramuscular, intraperitoneal, or subcutaneous, or administered proximal to the site of the target. The pharmaceutically acceptable carrier should be suitable for intravenous, intramuscular, subcutaneous, parenteral, intranasal, inhalational, spinal or epidermal administration (e.g., by injection or infusion). Depending on the route of administration, the active compound, e.g., antibody, may be coated in a material to protect the compound from the action of acids and other natural conditions that may inactivate the compound.

### Example

These examples are provided for illustrative purposes only and not to limit the scope of the invention.

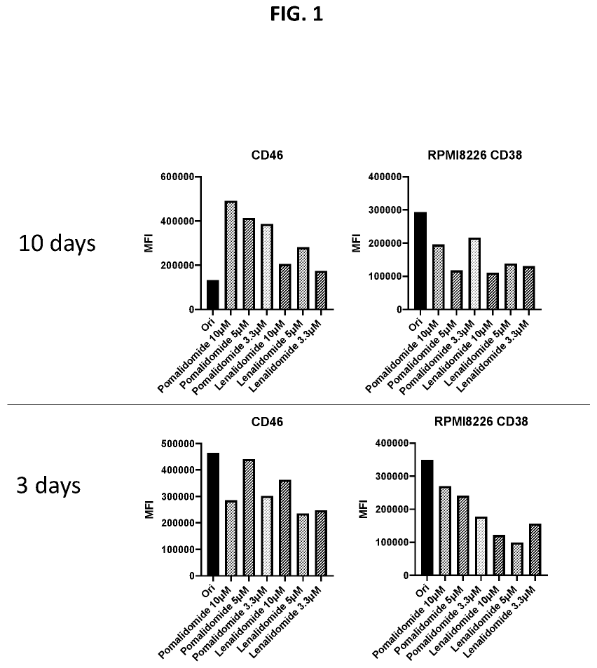

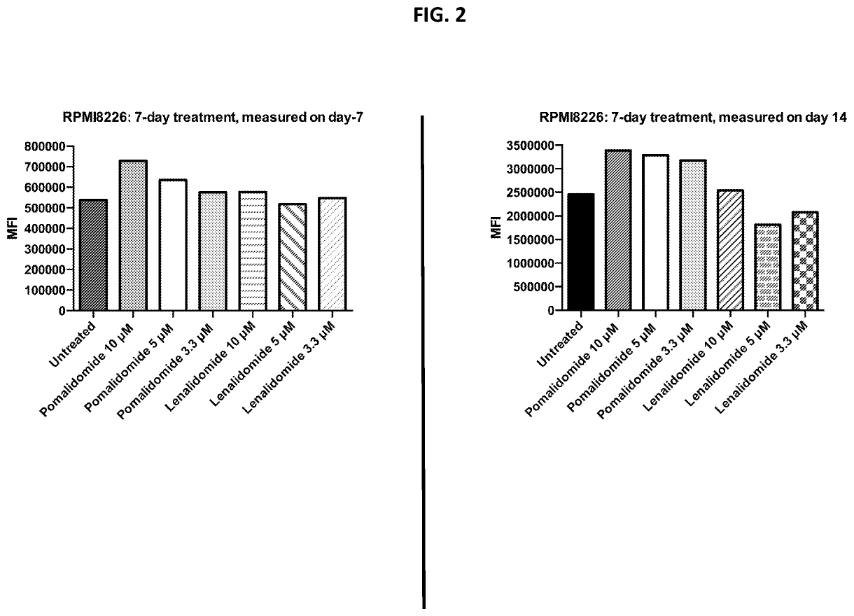

Example 1: CD46 upregulation by exposure to pomalidomide and lenalidomide (FIGS. 1-2). The effect of pomalidomide and lenalidomide exposure to multiple myeloma (RPMI8226) cells was studied. RPMI8226 cells were treated with IMiDs (Pomalidomide and Lenalidomide) for 3 and 10 days (FIG. 1). CD46 expression was analyzed by FACS. CD38 was also measured as a reference. IMiDs-induced cell surface upregulation was observed only to selective antigens, e.g., CD46 but not CD38.

FIG. 2 shows exposure to varying concentrations of pomalidomide and lenalidomide. The left portion displays CD46 upregulation on RPMI8226 cells following 7-day exposure to varying concentrations of pomalidomide and lenalidomide. FACS was performed on day-7. The right portion displays CD46 upregulation on RPMI8226 cells following a transient 7-day exposure to varying concentrations of pomalidomide and lenalidomide and 7-day recovery period. FACS was performed on day-14.

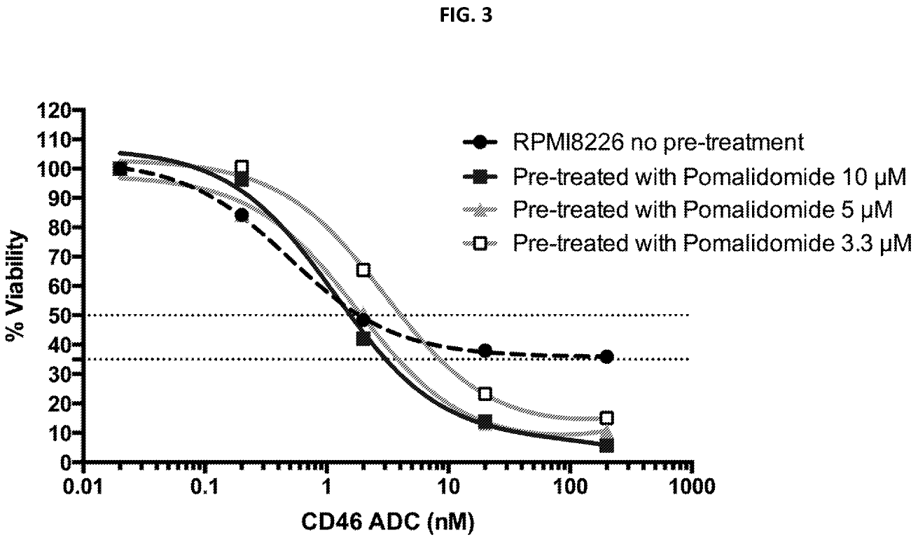

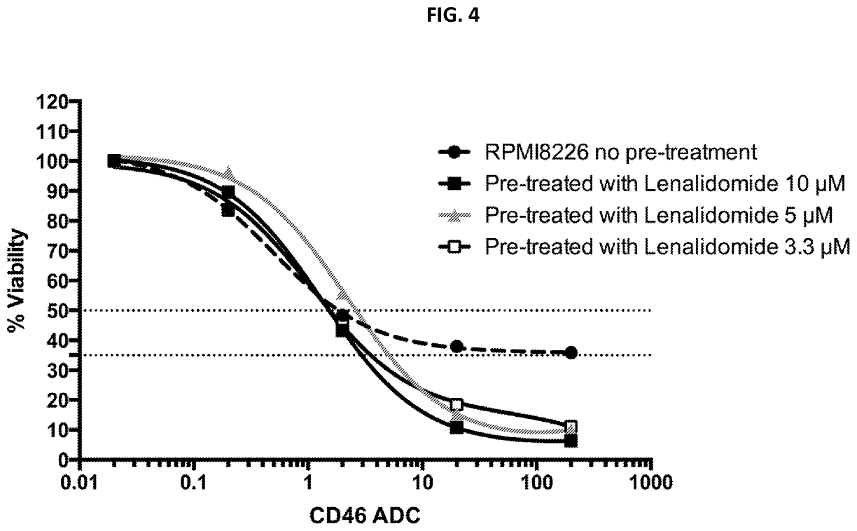

Example 2: Exploration of CD46 upregulation by IMiDs for enhanced efficacy by CD46 ADC (FIGS. 3-4). The effect of pomalidomide exposure, followed by CD46 ADC as a single agent, was examined. RPMI8226 cells seeded at 5,000 per well were exposed to 3.3 μM, 5 μM, and 10 μM pomalidomide for 7 days, washed, and treated with CD46 ADC. Cell viability was measured 96 h post ADC treatment. EC50 changed little, but EC65 (i.e., 35% viability) changed over 100-fold (no pre-treatment vs. 10 μM pom pre-treatment). The results are depicted in FIG. 3.

The effect of lenalidomide exposure, followed by CD46 ADC as a single agent, was also examined. RPMI8226 seeded at 5,000 per well was exposed to 3.3 μM, 5 μM, 10 μM lenalidomide for 7 days, washed, and treated with CD46 ADC. Cell viability was measured 96 h post ADC treatment. EC50 changed little, but EC65 (i.e., 35% viability) changed over 100-fold (no pre-treatment vs. 10 μM len pre-treatment). The results are depicted in FIG. 4.

In summary, prior exposure to pomalidomide for a short period (7-10 days) led to rather persistent CD46 upregulation on multiple myeloma cell surface, resulting in enhanced potency of CD46 ADC as a single agent. Similar results were obtained for lenalidomide pre-exposure. It is expected that thalidomide and other thalidomide analogs will have similar activity as both pomalidomide and lenalidomide are analogs/derivatives of thalidomide.

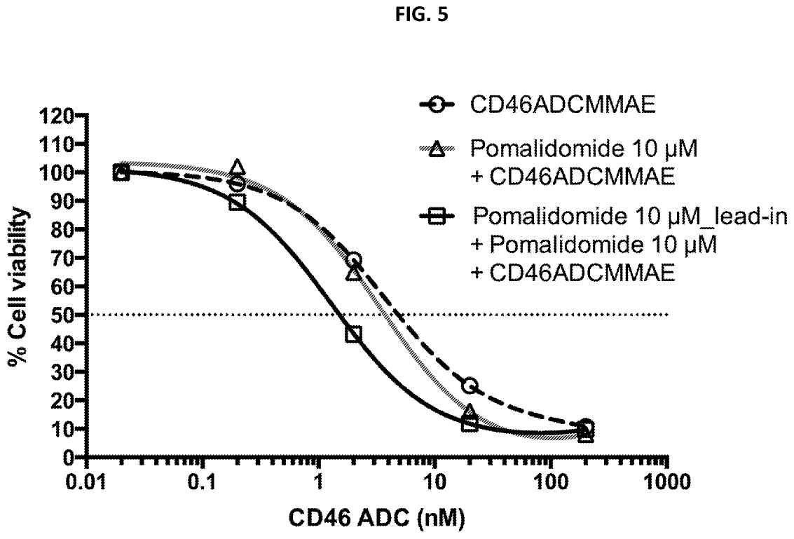

Example 3: Exposure to pomalidomide (pom), followed by combination treatment with pomalidomide plus CD46 ADC (FIG. 5). RPMI8226 cells seeded at 3,000 per well were exposed to 10 μM pomalidomide for 7 days, and treated with 10 μM pomalidomide plus CD46 ADC. Cell viability was measured 96 h post combination treatment. EC50=3.717 nM for CD46 ADC (single agent, no pretreatment with pom), 3.438 nM for pom+CD46 ADC combo (no pretreatment), and 1.297 nM for pom pretreatment followed by pom plus CD46 ADC combo.

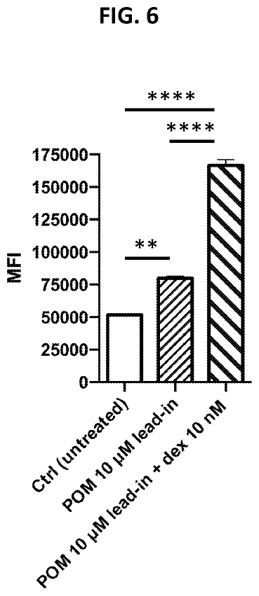

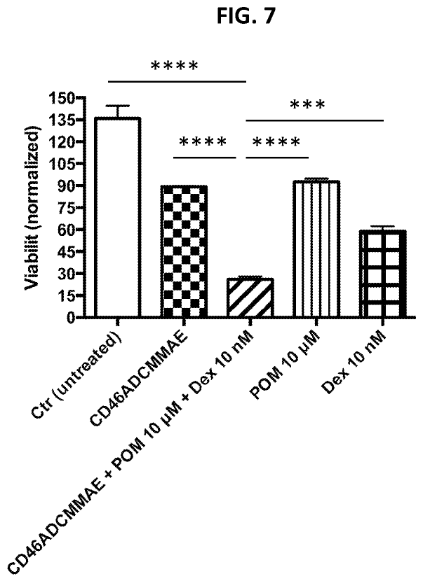

Example 4: Pomalidomide (pom) lead-in plus pom/dexamethasone (dex)/CD46 ADC combination further enhances CD46 expression (FIG. 6) and anti-tumor potency (FIG. 7).

FIG. 6: The multiple myeloma cell line RPMI8226 was incubated with 10 μM pomalidamide (pom) for 14 days, washed, and further incubated with 10 nM dexamethasone (dex) for 7 days. Cell surface CD46 expression was assessed by flow cytometry. MFI: median fluorescence intensity. Multiple group comparison was performed using One-way ANOVA. ** p<0.01; ****p<0.0001.

FIG. 7: RPMI8226 cells were exposed to 10 μM pomalidomide, followed by incubation with 0.2 nM CD46 ADC (CD46ADCMMAE), or 0.2 nM CD46 ADC+10 μM pom+10 nM dex (combo) for 4 days. Single treatments with 10 μM pom or 10 nM dex are also performed as controls. Cell viability was normalized and compared by One-Way ANOVA. Multiple comparison analysis was performed against the combo group. ***p<0.001; ****p<0.0001. Maximal cell killing was achieved with a combination of pom/dex/CD46 ADC.

Example 5:

Methods: RMPI8226 cells were exposed to varying concentrations of the Stat3 inhibitor C188-9 and the glucocorticoid receptor inhibitor mifepristone for 7 days at 37° C. in culture media, and cell surface CD46 expression is detected by flow cytometry using the anti-CD46 antibody YS5 followed by AlexaFluo-617 labeled anti-human Fc secondary antibody. MFI (median fluorescence intensity) was recorded and analyzed by One-way ANOVA.

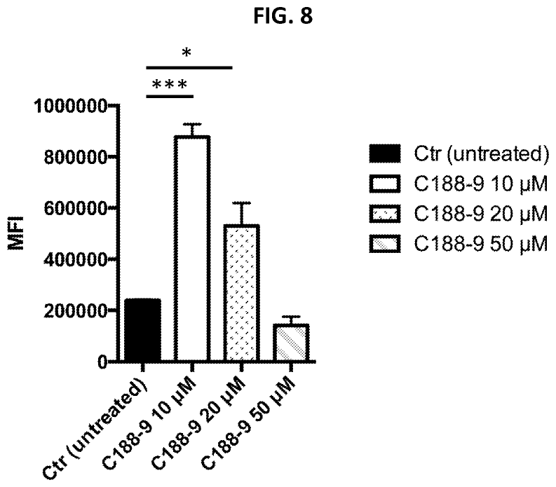

Finding: The Stat3 inhibitor C188-9 showed a concentration dependent effect on CD46 cell surface presentation on multiple myeloma cells: at low/mid concentrations (10 and 20 μM) it stimulates and at high concentrations (50 μM) it inhibits (FIG. 8). Stat3 has been reported to bind directly to the promoter region of the CD46 gene and upregulate CD46 expression. However, surprisingly, at lower concentrations a Stat3 inhibitor can up-regulate CD46 expression.

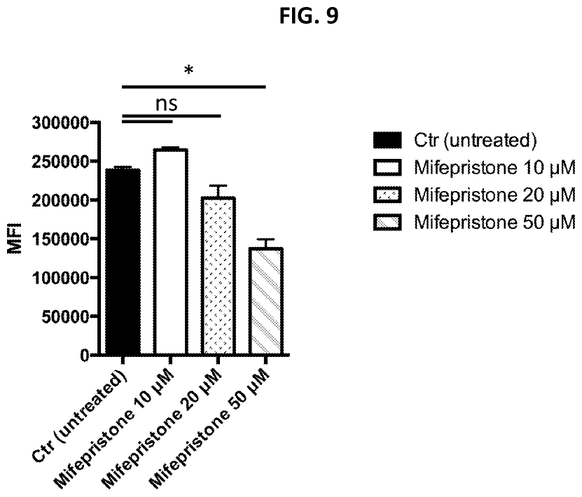

The glucocorticoid receptor inhibitor mifepristone inhibits CD46 cell surface presentation at high concentrations (50 μM) (FIG. 9). Inhibition of the glucocorticoid receptor at high concentrations supports the hypothesis that the glucocorticoid receptor signaling pathway is involved in CD46 regulation, providing complementary data to dexamethasone that is a glucocorticoid receptor agonist and upregulates CD46 in multiple myeloma cells.

The examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A method of treating multiple myeloma, lymphoma, acute myeloid leukemia (AML), prostate cancer or metastatic renal cell carcinoma (mRCC) in a human, the method comprising
administering to the human:
an antibody that specifically binds to CD46, wherein the antibody is linked to a cytotoxic effector; and
an agent that increases CD46 expression in cancer cells, wherein the agent is an immunomodulatory imide drug (IMid), optionally in combination with a glucocorticoid receptor agonist or modulator (SEGRAM),
wherein administration of the antibody and the agent kills more cancer cells than administration of the antibody alone.

2. The method of claim 1, wherein the agent is selected from the group consisting of pomalidamide, lenolidamide, thalidomide, iberdomide, and apremilast.

3. The method of any one of claims 1-2, wherein the administering comprises administering the agent without the antibody for a time sufficient to induce increased expression of CD46 in cancer cells followed by administering the antibody in an amount sufficient to kill myeloma cells in the human.

4. The method of claim 3, wherein administering the antibody further comprises administering an IMid, a SEGRAM, or both with the antibody.

5. The method of any one of claims 1-4, wherein the SEGRAM is selected from the group consisting of dexamethasone, prednisone, cortisol, mapracorat, fosdagrocorat (PF-04171327), and dagrocorat.

6. The method of any one of claims 3-4, wherein the time comprises 1-30 days (e.g., 2-20, or 3-15, 5-10 days) before administering the antibody.

7. The method of any one of claims 1-6, wherein the antibody comprises heavy chain CDRs 1, 2 and 3 and light chain CDRs 1, 2, and 3 of any one of YS5, YS5F, YS5vlD, SB1HGNY, YS12, 3G7RY (aka 3G8), YS6, YS1, YS3, YS4, YS8, YS7, YS9, YS10, YS11, 3G7HY, 3G7NY, 3G7, SB2, 2C8, or UA8kappa.

8. The method of any one of claims 1-6, wherein the antibody comprises a heavy chain (HC) variable region that comprises three complementarity determining regions (CDRs): HC CDR1, HC CDR2 and HC CDR3 and a light chain (LC) variable region that comprises three CDRs: LC CDR1, LC CDR2, and LC CDR3, wherein said HC CDR1, HC CDR2, HC CDR3 comprise an amino acid sequence of SEQ ID NO: 80, SEQ ID NO: 81, and SEQ ID NO: 82, respectively, and said LC CDR1, LC CDR2, and LC CDR3 comprise an amino acid sequence of SEQ ID NO: 83, SEQ ID NO: 84, and SEQ ID NO: 85, respectively.

9. The method of any one of claims 1-7, wherein the cytotoxic effector is a chemotherapeutic agent.

10. The method of any one of claims 1-7, wherein the cytotoxic effector is a microtubule inhibitor, a DNA-damaging agent, or a polymerase inhibitor.

11. The method of any one of claims 1-7, wherein the cytotoxic effector is selected from the group consisting of an auristatin, Dolastatin-10, synthetic derivatives of the natural product Dolastatin-10, and maytansine or a maytansine derivative.

12. The method of claim 11, wherein the cytotoxic effector is selected from the group consisting Monomethylauristatin F (MMAF), Auristatin E (AE), Monomethylauristatin E (MMAE), vcMMAE, and vcMMAF.

13. The method of any one of claims 1-6, wherein the antibody comprises a heavy chain (HC) variable region that comprises three complementarity determining regions (CDRs): HC CDR1, HC CDR2 and HC CDR3 and a light chain (LC) variable region that comprises three CDRs: LC CDR1, LC CDR2, and LC CDR3, wherein said HC CDR1, HC CDR2, HC CDR3 comprise an amino acid sequence of SEQ ID NO: 80, SEQ ID NO: 81, and SEQ ID NO: 82, respectively, and said LC CDR1, LC CDR2, and LC CDR3 comprise an amino acid sequence of SEQ ID NO: 83, SEQ ID NO: 84, and SEQ ID NO: 85, respectively; and
(b) monomethylauristatin E (MMAE) that is conjugated to said antibody via a maleimidocaproyl-valine-citrulline-para-amino benzyloxycarbonyl (mc-vc-PAB) linker.

14. The method of claim 13, wherein the HC comprises SEQ ID NO:86 and the LC comprises SEQ ID NO:87.

15. A pharmaceutical composition comprising
an anti-CD46 antibody conjugated to a cytotoxic effector; and
an agent that is an immunomodulatory imide drug (IMid) that increases CD46 expression in cancer cells, optionally in combination with a glucocorticoid receptor agonist or modulator (SEGRAM).

16. The pharmaceutical composition of claim 15, wherein the agent is selected from the group consisting of pomalidamide, lenolidamide, thalidomide, iberdomide, and apremilast.

17. The pharmaceutical composition of claim 15, the SEGRAM is selected is selected from the group consisting of dexamethasone, prednisone, cortisol, mapracorat, fosdagrocorat (PF-04171327), and dagrocorat.

18. The pharmaceutical composition of any one of claims 15-17, wherein the antibody comprises heavy chain CDRs 1, 2 and 3 and light chain CDRs 1, 2, and 3 of any one of YS5, YS5F, YS5vlD, SB1HGNY, YS12, 3G7RY (aka 3G8), YS6, YS1, YS3, YS4, YS8, YS7, YS9, YS10, YS11, 3G7HY, 3G7NY, 3G7, SB2, 2C8, or UA8kappa.

19. The pharmaceutical composition of any one of claims 15-17, wherein the antibody comprises a heavy chain (HC) variable region that comprises three complementarity determining regions (CDRs): HC CDR1, HC CDR2 and HC CDR3 and a light chain (LC) variable region that comprises three CDRs: LC CDR1, LC CDR2, and LC CDR3, wherein said HC CDR1, HC CDR2, HC CDR3 comprise an amino acid sequence of SEQ ID NO: 80, SEQ ID NO: 81, and SEQ ID NO: 82, respectively, and said LC CDR1, LC CDR2, and LC CDR3 comprise an amino acid sequence of SEQ ID NO: 83, SEQ ID NO: 84, and SEQ ID NO: 85, respectively.

20. The pharmaceutical composition of any one of claims 15-19, wherein the cytotoxic effector is a chemotherapeutic agent.

21. The pharmaceutical composition of any one of claims 15-19, wherein the cytotoxic effector is a microtubule inhibitor, a DNA-damaging agent, or a polymerase inhibitor.

22. The pharmaceutical composition of any one of claims 15-19, wherein the cytotoxic effector is selected from the group consisting of an auristatin, Dolastatin-10, synthetic derivatives of the natural product Dolastatin-10, and maytansine or a maytansine derivative.

23. The pharmaceutical composition of claim 22, wherein the cytotoxic effector is selected from the group consisting Monomethylauristatin F (MMAF), Auristatin E (AE), Monomethylauristatin E (MMAE), vcMMAE, and vcMMAF.

24. The pharmaceutical composition of any one of claims 15-16, wherein the antibody comprises a heavy chain (HC) variable region that comprises three complementarity determining regions (CDRs): HC CDR1, HC CDR2 and HC CDR3 and a light chain (LC) variable region that comprises three CDRs: LC CDR1, LC CDR2, and LC CDR3, wherein said HC CDR1, HC CDR2, HC CDR3 comprise an amino acid sequence of SEQ ID NO: 80, SEQ ID NO: 81, and SEQ ID NO: 82, respectively, and said LC CDR1, LC CDR2, and LC CDR3 comprise an amino acid sequence of SEQ ID NO: 83, SEQ ID NO: 84, and SEQ ID NO: 85, respectively; and
(b) monomethylauristatin E (MMAE) that is conjugated to said antibody via a maleimidocaproyl-valine-citrulline-para-amino benzyloxycarbonyl (mc-vc-PAB) linker.

25. The pharmaceutical composition of claim 24, wherein the HC comprises SEQ ID NO:86 and the LC comprises SEQ ID NO:87.

26. A method of treating multiple myeloma, lymphoma, acute myeloid leukemia (AML), prostate cancer or metastatic renal cell carcinoma (mRCC) in a human, the method comprising
administering to the human:
an antibody that specifically binds to CD46, wherein the antibody is linked to a cytotoxic effector; and
an agent that increases CD46 expression in cancer cells, wherein the agent is a Signal Transducer And Activator of Transcription 3 (STAT3) inhibitor, optionally in combination with an immunomodulatory imide drug (IMiD) and/or a glucocorticoid receptor agonist or modulator (SEGRAM) or both,
wherein administration of the antibody and the agent kills more cancer cells than administration of the antibody alone.

27. The method of claim 26, wherein the agent is selected from the group consisting of N-(1′, 2-Dihydroxy-1,2′-binaphthalen-4′-yl)-4-methoxybenzenesulfonamide (C188-9), STAT3 Inhibitor V, 6-Nitrobenzo[b]thiophene 1,1-dioxide (Stattic), (1E,6E)-1,7-Bis(4-hydroxy-3-methoxyphenyl)-1,6-heptadiene-3,5-dione (curcumin), N-Hexyl-2-(1-naphthalenyl)-5-[[4-(phosphonooxy)phenyl]methyl]-4-oxazolecarboxamide (S3I-M2001), 8-hydroxy-3-methyl-3,4-dihydrotetraphene-1,7,12(2H)-trione (STA-21), 2-Hydroxy-4-[[2-[[(4-methylphenyl)sulfonyl]oxy]acetyl]amino]benzoic acid (S3I-201), Cepharanthine, Cucurbitacin I, Cucumis sativus L, Niclosamide, Cryptotanshinone, SD 1008, Stat3 Inhibitor III, WP1066, Nifuroxazide, Stat3 Inhibitor VI, S3I-201, STA-21, Kahweol, STAT3 Inhibitor IX, Cpd188; STAT3 Inhibitor VI, S31-201; STAT3 Inhibitor VII Ethyl-1-(4-cyano-2,3,5,6-tetrafluorophenyl)-6,7,8-trifluoro-4-oxo-1,4-dih-ydroquinoline-3-carboxylate; STAT3 Inhibitor VIII, 5,15-DPP, STAT3 Inhibitor X, HJB; STAT3 Inhibitor XII, SPI; STAT3 Inhibitor XI, STX-0119; STAT3 Inhibitor XIV, LLL12; FLLL32; FLLL62; Napabucasin (BBI608); DSP-0337 (prodrug of napabucasin); OPB-51602; OPB-31121; OPB-111077; Pyrimethamine; WP1066 and derivatives or analogues thereof.

28. The method of any one of claims 26-27, wherein the administering comprises administering the agent without the antibody for a time sufficient to induce increased expression of CD46 in cancer cells followed by administering the antibody in an amount sufficient to kill myeloma cells in the human.

29. The method of claim 28, wherein administering the antibody further comprises administering the agent, a SEGRAM, or both with the antibody.

30. The method of any one of claims 26-29, wherein the SEGRAM is selected from the group consisting of dexamethasone, prednisone, cortisol, mapracorat, fosdagrocorat (PF-04171327), and dagrocorat.

31. The method of any one of claims 28-29, wherein the time comprises 1-30 days (e.g., 2-20, or 3-15, 5-10 days) before administering the antibody.

32. The method of any one of claims 26-31, wherein the antibody comprises heavy chain CDRs 1, 2 and 3 and light chain CDRs 1, 2, and 3 of any one of YS5, YS5F, YS5vlD, SB1HGNY, YS12, 3G7RY (aka 3G8), YS6, YS1, YS3, YS4, YS8, YS7, YS9, YS10, YS11, 3G7HY, 3G7NY, 3G7, SB2, 2C8, or UA8kappa.

33. The method of any one of claims 26-31, wherein the antibody comprises a heavy chain (HC) variable region that comprises three complementarity determining regions (CDRs): HC CDR1, HC CDR2 and HC CDR3 and a light chain (LC) variable region that comprises three CDRs: LC CDR1, LC CDR2, and LC CDR3, wherein said HC CDR1, HC CDR2, HC CDR3 comprise an amino acid sequence of SEQ ID NO: 80, SEQ ID NO: 81, and SEQ ID NO: 82, respectively, and said LC CDR1, LC CDR2, and LC CDR3 comprise an amino acid sequence of SEQ ID NO: 83, SEQ ID NO: 84, and SEQ ID NO: 85, respectively.

34. The method of any one of claims 26-33, wherein the cytotoxic effector is a chemotherapeutic agent.

35. The method of any one of claims 26-33, wherein the cytotoxic effector is a microtubule inhibitor, a DNA-damaging agent, or a polymerase inhibitor.

36. The method of any one of claims 26-33, wherein the cytotoxic effector is selected from the group consisting of an auristatin, Dolastatin-10, synthetic derivatives of the natural product Dolastatin-10, and maytansine or a maytansine derivative.

37. The method of claim 36, wherein the cytotoxic effector is selected from the group consisting Monomethylauristatin F (MMAF), Auristatin E (AE), Monomethylauristatin E (MMAE), vcMMAE, and vcMMAF.

38. The method of any one of claims 26-33, wherein the antibody comprises a heavy chain (HC) variable region that comprises three complementarity determining regions (CDRs): HC CDR1, HC CDR2 and HC CDR3 and a light chain (LC) variable region that comprises three CDRs: LC CDR1, LC CDR2, and LC CDR3, wherein said HC CDR1, HC CDR2, HC CDR3 comprise an amino acid sequence of SEQ ID NO: 80, SEQ ID NO: 81, and SEQ ID NO: 82, respectively, and said LC CDR1, LC CDR2, and LC CDR3 comprise an amino acid sequence of SEQ ID NO: 83, SEQ ID NO: 84, and SEQ ID NO: 85, respectively; and
(b) monomethylauristatin E (MMAE) that is conjugated to said antibody via a maleimidocaproyl-valine-citrulline-para-amino benzyloxycarbonyl (mc-vc-PAB) linker.

39. The method of claim 38, wherein the HC comprises SEQ ID NO:86 and the LC comprises SEQ ID NO:87.

40. A pharmaceutical composition comprising
an anti-CD46 antibody conjugated to a cytotoxic effector; and
an agent that is a Signal Transducer And Activator of Transcription 3 (STAT3) inhibitor at a concentration sufficient to increase CD46 expression in cancer cells, optionally in combination with a glucocorticoid receptor agonist or modulator (SEGRAM).

41. The pharmaceutical composition of claim 40, wherein the agent is selected from the group consisting of N-(1′, 2-Dihydroxy-1,2′-binaphthalen-4′-yl)-4-methoxybenzenesulfonamide (C188-9), STAT3 Inhibitor V, 6-Nitrobenzo[b]thiophene 1,1-dioxide (Stattic), (1E,6E)-1,7-Bis(4-hydroxy-3-methoxyphenyl)-1,6-heptadiene-3,5-dione (curcumin), N-Hexyl-2-(1-naphthalenyl)-5-[[4-(phosphonooxy)phenyl]methyl]-4-oxazolecarboxamide (S3I-M2001), 8-hydroxy-3-methyl-3,4-dihydrotetraphene-1,7,12(2H)-trione (STA-21), 2-Hydroxy-4-[[2-[[(4-methylphenyl)sulfonyl]oxy]acetyl]amino]benzoic acid (S3I-201), Cepharanthine, Cucurbitacin I, Cucumis sativus L, Niclosamide, Cryptotanshinone, SD 1008, Stat3 Inhibitor III, WP1066, Nifuroxazide, Stat3 Inhibitor VI, S3I-201, STA-21, Kahweol, STAT3 Inhibitor IX, Cpd188; STAT3 Inhibitor VI, S31-201; STAT3 Inhibitor VII Ethyl-1-(4-cyano-2,3,5,6-tetrafluorophenyl)-6,7,8-trifluoro-4-oxo-1,4-dih-ydroquinoline-3-carboxylate; STAT3 Inhibitor VIII, 5,15-DPP, STAT3 Inhibitor X, HJB; STAT3 Inhibitor XII, SPI; STAT3 Inhibitor XI, STX-0119; STAT3 Inhibitor XIV, LLL12; FLLL32; FLLL62; Napabucasin (BBI608); DSP-0337 (prodrug of napabucasin); OPB-51602; OPB-31121; OPB-111077; Pyrimethamine; WP1066 and derivatives or analogues thereof.

42. The pharmaceutical composition of claim 40, the SEGRAM is selected is selected from the group consisting of dexamethasone, prednisone, cortisol, mapracorat, fosdagrocorat (PF-04171327), and dagrocorat.

43. The pharmaceutical composition of any one of claims 40-42, wherein the antibody comprises heavy chain CDRs 1, 2 and 3 and light chain CDRs 1, 2, and 3 of any one of YS5, YS5F, YS5vlD, SB1HGNY, YS12, 3G7RY (aka 3G8), YS6, YS1, YS3, YS4, YS8, YS7, YS9, YS10, YS11, 3G7HY, 3G7NY, 3G7, SB2, 2C8, or UA8kappa.

44. The pharmaceutical composition of any one of claims 40-42, wherein the antibody comprises a heavy chain (HC) variable region that comprises three complementarity determining regions (CDRs): HC CDR1, HC CDR2 and HC CDR3 and a light chain (LC) variable region that comprises three CDRs: LC CDR1, LC CDR2, and LC CDR3, wherein said HC CDR1, HC CDR2, HC CDR3 comprise an amino acid sequence of SEQ ID NO: 80, SEQ ID NO: 81, and SEQ ID NO: 82, respectively, and said LC CDR1, LC CDR2, and LC CDR3 comprise an amino acid sequence of SEQ ID NO: 83, SEQ ID NO: 84, and SEQ ID NO: 85, respectively.

45. The pharmaceutical composition of any one of claims 40-44, wherein the cytotoxic effector is a chemotherapeutic agent.

46. The pharmaceutical composition of any one of claims 40-44, wherein the cytotoxic effector is a microtubule inhibitor, a DNA-damaging agent, or a polymerase inhibitor.

47. The pharmaceutical composition of any one of claims 40-44, wherein the cytotoxic effector is selected from the group consisting of an auristatin, Dolastatin-10, synthetic derivatives of the natural product Dolastatin-10, and maytansine or a maytansine derivative.

48. The pharmaceutical composition of claim 47, wherein the cytotoxic effector is selected from the group consisting Monomethylauristatin F (MMAF), Auristatin E (AE), Monomethylauristatin E (MMAE), vcMMAE, and vcMMAF.

49. The pharmaceutical composition of any one of claims 40-42, wherein the antibody comprises
(a) a heavy chain (HC) variable region that comprises three complementarity determining regions (CDRs): HC CDR1, HC CDR2 and HC CDR3 and a light chain (LC) variable region that comprises three CDRs: LC CDR1, LC CDR2, and LC CDR3, wherein said HC CDR1, HC CDR2, HC CDR3 comprise an amino acid sequence of SEQ ID NO: 80, SEQ ID NO: 81, and SEQ ID NO: 82, respectively, and said LC CDR1, LC CDR2, and LC CDR3 comprise an amino acid sequence of SEQ ID NO: 83, SEQ ID NO: 84, and SEQ ID NO: 85, respectively; and
(b) monomethylauristatin E (MMAE) that is conjugated to said antibody via a maleimidocaproyl-valine-citrulline-para-amino benzyloxycarbonyl (mc-vc-PAB) linker.

50. The pharmaceutical composition of claim 49, wherein the HC comprises SEQ ID NO:86 and the LC comprises SEQ ID NO:87.
